(19) United States
(12) Patent Application Publication
(10) Pub. No.: US 2019/0062441 A1
(43) Pub. Date: Feb. 28, 2019

(54) Methods For Treating Rare Genetic Disorders Using Glucagon Receptor Antagonistic Antibodies

(71) Applicant: REMD Biotherapeutics, Inc., Camarillo, CA (US)

(72) Inventors: Hai Yan, Thousand Oaks, CA (US); Jeong Oh, Newbury Park, CA (US)

(21) Appl. No.: 15/766,821

(22) PCT Filed: Oct. 6, 2016
(86) PCT No.: PCT/US16/55864

§ 371 Date: Apr. 8, 2018

Related U.S. Application Data
(60) Provisional application No. 62/238,290, filed on Oct. 7, 2015.

(51) Int. Cl. C07K 16/28; A61P 3/10; A61K 45/06; A61K 39/395

(52) U.S. Cl. CPC C07K 16/2869; A61P 3/10; A61K 45/06; A61K 2039/505; C07K 2317/24; C07K 2317/21; C07K 2317/76; A61K 39/3955

(57) ABSTRACT

The present disclosure relates to methods for treating various rare genetic disorders which are characterized, in part, by abnormal resistance to insulin and/or hyperglycemia using a glucagon receptor blocking agent. In various embodiments, the present disclosure relates to methods for treating a rare genetic disorder using antigen binding and antagonizing proteins, e.g., fully human antibodies, that specifically bind to and antagonize the function of the human glucagon receptor.

RELATED PATENT APPLICATIONS

This application claims benefit of U.S. Provisional Application No. 62/238,290, filed on Oct. 7, 2015, incorporated by reference in its entirety.

### TECHNICAL FIELD

The human body requires that the blood glucose level be maintained in a very narrow range. Circulating glucose and metabolic homeostasis are regulated by the equally important, opposing actions of insulin and glucagon (Unger and Cherrington, J Clin Invest., 122:4-12, 2012). Under normal circumstances, the body is able to balance the amount of glucose, or sugar, in the blood with the amount of glucose that the cells need for fuel (glucose homeostasis). The hormone insulin, which is produced in beta cells in the pancreas affects the transport of glucose into the cells. Loss of glucose homeostasis typically results in metabolic disorders such as obesity, diabetes and hyperglycemia. People with diabetes have high blood glucose, also called high blood sugar or hyperglycemia. In people with type 1 diabetes (T1 D), the body does not produce enough insulin due to the destruction of beta cells in the pancreas, which results in the lack of suppression of glucagon action. Type 2 diabetes (T2D) is caused by a combination of factors, including insulin resistance, a condition in which the body's muscle, fat, and liver cells do not use insulin effectively and the body can no longer produce enough insulin to compensate for the impaired ability to use insulin.

Activation of the hepatic glucagon receptor (GCGR) is directly responsible for decreased hepatic glucose uptake, increased hepatic glycogenolysis, increased hepatic gluconeogenesis, increased ketogenesis, and decreased glycogen synthesis, all of which increase circulating glucose (Unger and Orci, Lancet, 1:14-16, 1975). The main target organ of glucagon is the liver where GCGR is expressed at uniquely high levels. Activation of GCGR in hepatocytes stimulates the synthesis and the biochemical activity of key enzymes for glycogenolysis and gluconeogenesis resulting in increased hepatic glucose output. It has been demonstrated that targeting glucagon production or function using isolated antagonistic antigen binding proteins that specifically bind to and antagonize the human GCGR are capable of controlling and lowering blood glucose, and improving glucose tolerance, in T2D models (see, e.g., U.S. Pat. No. 7,947,809 (Yan, et al)); and capable of normalizing blood glucose and hemoglobin A1c levels in the complete absence of insulin therapy, in T1 D models (see, e.g., PCT WO 2015/189698 (Yan and Shi)).

There are a number of extremely rare genetic disorders which are characterized, in part, by abnormal resistance to insulin and/or hyperglycemia. Such disorders include, e.g., Maturity Onset Diabetes of the Young Syndrome (MODY), Wolfram Syndrome, Neonatal Diabetes, Maternally Inherited Diabetes and Deafness Syndrome, Prader-Willi Syndrome, Rabson-Mendenhall Syndrome, Insulin-resistant Acanthosis Nigricans (IRAN), Type A, Leprechaunism, Lipodystrophies, Fanconi Bickel Syndrome (FBS), Friedreich Ataxia, Klinefelter's Syndrome, Laurence-Moon Syndrome, and a class of rare genetic disorders referred to as ‘ciliopathies’, including e.g., Alström syndrome (ALMS), Bardet-Biedl syndrome (BBS), nephronophthisis (NPHP), Senior-Löken syndrome (SNLS), Meckel syndrome (MKS), Joubert syndrome (JBTS), Oral-facial-digital Type I (OFD 1), Jeune asphyxiating thoracic dystrophy (JATD), Ellis van Creveld (EVC), Leber congenital amaurosis (LCA), and polycystic kidney diseases (PKD).

Alstrom syndrome (ALMS) is a very rare autosomal-recessive disorder caused solely by mutations in ALMS1. Symptoms of ALMS include childhood obesity (95% of patients), type 2 diabetes (T2D), retinal degeneration, cardiomyopathy, renal impairment, infertility and sensorineuronal hearing loss (Girard and Petrovsky, Nat. Rev. Endocrinol. 7:77-88, 2011). Within the first 5 years of life, patients with ALMS develop truncal obesity, insulin resistance, hyperglycemia, hyperleptinemia, hyperinsulinemia, hyperlipidemia, which progress to clinical T2D. Mouse models of ALMS also demonstrate most of the clinical symptoms of ALMS seen in human patients, including obesity, hyperglycemia, hyperinsulinemia, hyperlipidemia, steatohepatitis, infertility, and sensorineuronal hearing loss.

In view of the fact that obesity, hyperglycemia, and T2D are associated with ALMS and various other rare genetic disorders, research efforts to develop novel treatments and/or new methods of managing or ameliorating the clinical symptoms of these rare genetic disorders could benefit greatly from studies evaluating the effects of such antagonistic antigen binding proteins to effectively treat patients who have, or who have been diagnosed with any such disorders.

### DISCLOSURE OF THE INVENTION

The present disclosure is based, in part, on the inventor's unique insight that isolated antigen binding and antagonizing proteins that specifically bind to the human glucagon receptor may provide for improved, effective methods of treating various rare genetic disorders which are characterized, in part, by abnormal resistance to insulin and/or hyperglycemia and/or provide for improved, effective methods for managing and/or ameliorating various clinical symptoms of such disorders. Specifically, the present inventors propose that the beneficial therapeutic effects provided by regulating endogenous glucose output (via blocking the glucagon receptor) in subjects diagnosed with such disorders may include: reduction in weight gain; reduction of blood glucose levels in vivo; preventing hyperglycemia; preventing hyperglucagonemia; reducing insulin insufficiency; reducing hyperinsulinemia; alleviating complications associated with insulin monotherapy, including insulin treatment induced hypoglycemia; reducing or preventing hyperinsulinemia, reducing or preventing fat deposits in the liver; reducing or preventing inflammation in the liver; reducing or preventing the accumulation of lipid, e.g., hepatic triacylglycerol, hepatic diacylglycerol, and ceramides; and preventing injury in the liver.

Thus, in one aspect, the present disclosure comprises a method for treating a rare genetic disorder in a subject, comprising administering to the subject a therapeutically effective amount of an isolated antagonistic antigen binding protein that specifically binds to the human glucagon receptor.

In various embodiments, the rare genetic disorder is a disorder associated with, or characterized, in part, by abnormal resistance to insulin and/or hyperglycemia. In various embodiments, the rare genetic disorder is a ciliopathy selected from the group consisting of Bardet-Biedl syndrome (BBS), nephronophthisis (NPHP), Senior-Löken syndrome (SNLS), Alström syndrome (ALMS), Meckel syndrome (MKS), Joubert syndrome (JBTS), Oral-facial-digital Type I (OFD 1), Jeune asphyxiating thoracic dystrophy (JATD), Ellis van Creveld (EVC), Leber congenital amaurosis (LCA), autosomal recessive polycystic kidney disease (ARPKD) and autosomal dominant polycystic kidney disease (ADPKD). In various embodiments, the rare genetic disorder is selected from the group consisting of Maturity Onset Diabetes of the Young Syndrome (MODY), Wolfram Syndrome, Neonatal Diabetes, Maternally Inherited Diabetes and Deafness Syndrome, Prader-Willi Syndrome, Rabson-Mendenhall Syndrome, Insulin-resistant Acanthosis Nigricans (IRAN), Leprechaunism, Lipodystrophies, Fanconi Bickel Syndrome (FBS), Friedreich Ataxia, Klinefelter's Syndrome, Laurence-Moon Syndrome. In various embodiments, the rare genetic disorder is Maturity Onset Diabetes of the Young Syndrome (MODY). In various embodiments, the rare genetic disorder is Rabson-Mendenhall Syndrome. In various embodiments, the rare genetic disorder is Wolfram Syndrome. In various embodiments, the ciliopathy is Alstrom syndrome (ALMS). In various embodiments, the ciliopathy is Bardet-Biedl syndrome (BBS). In various embodiments, the rare genetic disorder is Neonatal Diabetes. In various embodiments, the rare genetic disorder is Prader-Willi Syndrome.

In another aspect, the present disclosure comprises a method for managing and/or ameliorating one or more symptoms associated with a rare genetic disorder in a subject, comprising administering to the subject a therapeutically effective amount of an isolated antagonistic antigen binding protein that specifically binds to the human glucagon receptor. In various embodiments, the rare genetic disorder is a disorder characterized, in part, by abnormal resistance to insulin and/or hyperglycemia. In various embodiments, the one or more symptoms is selected from the group consisting of obesity, acute or chronic hyperglycemia, hyperinsulinemia, hyperlipidemia, steatohepatitis, excess gluconeogenesis, excess glycogenolysis, ketosis, diabetic ketoacidosis, hypertriglyceridemia, elevated plasma free fatty acids, hypertension, diabetic nephropathy, renal insufficiency, renal failure, hyperphagia, diabetic neuropathy, diabetic retinopathy, or diabetic coma, excess HbA1c levels, polyuria (frequent urination), polydipsia (increased thirst), xerostomia (dry mouth), polyphagia (increased hunger), fatigue, and kidney dialysis. In various embodiments, the symptom is acute or chronic hyperglycemia. In various embodiments, the symptom is hyperglucagonemia.

In various embodiments, the isolated antagonistic binding protein binds to a human glucagon receptor with a dissociation constant (K_{D}) of at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

In various embodiments, the isolated antagonistic antigen binding protein comprises an antibody selected from a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′2, a IgG, a IgM, a IgA, a IgE, a scFv, a dsFv, a dAb, a nanobody, a unibody, or a diabody. In various embodiments, the antibody is a fully human monoclonal antibody.

In various embodiments, the isolated antagonistic binding protein is a fully human antibody which comprises the amino acid sequence encoding the heavy chain variable region of SEQ ID NO: 2 and the amino acid sequence encoding the light chain variable region of SEQ ID NO: 3.

In various embodiments, the isolated antagonistic protein is a fully human antibody which comprises the amino acid sequence encoding the heavy chain variable region of SEQ ID NO: 4 and the amino acid sequence encoding the light chain variable region of SEQ ID NO: 5.

In various embodiments, the isolated antagonistic protein is a fully human antibody which comprises the amino acid sequence encoding the heavy chain variable region of SEQ ID NO: 6 and the amino acid sequence encoding the light chain variable region of SEQ ID NO: 7.

In various embodiments, the isolated antagonistic protein is a fully human antibody which comprises a heavy chain variable region having the amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28.

In various embodiments, the isolated antagonistic protein is a fully human antibody which comprises a light chain variable region having the amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47.

In various embodiments, the isolated antagonistic protein is a fully human antibody which comprises the amino acid sequence encoding the heavy chain of SEQ ID NO: 51 and the amino acid sequence encoding the light chain of SEQ ID NO: 52.

In another aspect, the present disclosure comprises a method for treating a rare genetic disorder in a subject, comprising administering to the subject (a) a therapeutically effective amount of an isolated antagonistic antigen binding protein that specifically binds to the human glucagon receptor, and (b) a second therapeutic agent. In various embodiments, the combination of isolated antagonistic antigen binding protein and second therapeutic agent has a synergistic effect. In various embodiments, the second therapeutic agent is a glucose lowering agent selected from the group consisting of anti-diabetic agents, anti-hyperglycemic agents, lipid lowering agents, and anti-hypertensive agents. In various embodiments, the second therapeutic agent is an anti-obesity agent selected from the group consisting of gut-selective MTP inhibitors, CCKa agonists, 5HT2c agonists, MCR4 agonists, lipase inhibitors, opioid antagonists, oleoyl-estrone, obinepitide, pramlintide (SYMLIN®), tesofensine, leptin, bromocriptine, orlistat, AOD-9604, and sibutramine.

In another aspect, the present disclosure relates to the use of a non-naturally occurring isolated antagonistic antigen binding protein that specifically binds to the human glucagon receptor for the preparation of a medicament for treatment of a rare genetic disorder in a subject in need thereof.

In various embodiments, the isolated antagonistic antigen binding protein that specifically binds the human glucagon receptor will be admixed with a pharmaceutically acceptable carrier to form a pharmaceutical composition that can be systemically administered to the subject via intravenous injection, intramuscular injection, subcutaneous injection, intraperitoneal injection, transdermal injection, intra-arterial injection, intrasternal injection, intrathecal injection, intraventricular injection, intraurethral injection, intracranial injection, intrasynovial injection or via infusions.

### MODE(S) FOR CARRYING OUT THE INVENTION

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present disclosure are generally performed according to conventional methods well known in the art and as described in various general and more specific references that are cited and discussed throughout the present specification unless otherwise indicated. See, e.g., Sambrook et al. Molecular Cloning: A Laboratory Manual, 2nd ed., Cold Spring Harbor Laboratory Press, Cold Spring Harbor, N. Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N. Y. (1990), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

### Definitions

The terms “peptide” “polypeptide” and “protein” each refers to a molecule comprising two or more amino acid residues joined to each other by peptide bonds. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. In certain embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free a-amino group on an amino acid at the amino terminal of a peptide or to the a-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

The term “therapeutic protein” refers to proteins, polypeptides, antibodies, peptides or fragments or variants thereof, having one or more therapeutic and/or biological activities. Therapeutic proteins encompassed by the invention include but are not limited to, proteins, polypeptides, peptides, antibodies, and biologics. (The terms peptides, proteins, and polypeptides are used interchangeably herein.) It is specifically contemplated that the term “Therapeutic protein” encompasses antibodies and fragments and variants thereof.

Polynucleotide and polypeptide sequences are indicated using standard one- or three-letter abbreviations. Unless otherwise indicated, polypeptide sequences have their amino termini at the left and their carboxy termini at the right, and single-stranded nucleic acid sequences, and the top strand of double-stranded nucleic acid sequences, have their 5′ termini at the left and their 3′ termini at the right. A particular section of a polypeptide can be designated by amino acid residue number such as amino acids 80 to 119, or by the actual residue at that site such as Ser80 to Ser119. A particular polypeptide or polynucleotide sequence also can be described by explaining how it differs from a reference sequence.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:
Alanine (A), Serine (S), and Threonine (T)
Aspartic acid (D) and Glutamic acid (E)
Asparagine (N) and Glutamine (Q)
Arginine (R) and Lysine (K)
Isoleucine (I), Leucine (L), Methionine (M), and Valine (V)
Phenylalanine (F), Tyrosine (Y), and Tryptophan (W)

A “non-conservative amino acid substitution” refers to the substitution of a member of one of these classes for a member from another class. In making such changes, according to certain embodiments, the hydropathic index of amino acids may be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (see, for example, Kyte et al., 1982, J. Mol. Biol. 157:105-131). It is known that certain amino acids may be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those that are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as disclosed herein. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those that are within ±1 are included, and in certain embodiments, those within ±0.5 are included. Exemplary amino acid substitutions are set forth in Table 1.

TABLE 1

| Original Residues | Exemplary Substitutions | Preferred Substitutions |
|---|---|---|
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | |
| Trp | Tyr, Phe | Tyr |
| Tyr | Trp, Phe, Thr, Ser | Phe |
| Val | Ile, Met, Leu, Phe, | Leu |
| | Ala, Norleucine | |

A skilled artisan will be able to determine suitable variants of polypeptides as set forth herein using well-known techniques. In certain embodiments, one skilled in the art may identify suitable areas of the molecule that may be changed without destroying activity by targeting regions not believed to be important for activity. In other embodiments, the skilled artisan can identify residues and portions of the molecules that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a polypeptide that correspond to amino acid residues important for activity or structure in similar polypeptides. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three-dimensional structure. In certain embodiments, one skilled in the art may choose to not make radical changes to amino acid residues predicted to be on the surface of the polypeptide, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

The term “polypeptide fragment” and “truncated polypeptide” as used herein refers to a polypeptide that has an amino-terminal and/or carboxy-terminal deletion as compared to a corresponding full-length protein. In certain embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In certain embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant” and “polypeptide mutant” as used herein refers to a polypeptide that comprises an amino acid sequence wherein one or more amino acid residues are inserted into, deleted from and/or substituted into the amino acid sequence relative to another polypeptide sequence. In certain embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include fusion proteins.

A “derivative” of a polypeptide is a polypeptide that has been chemically modified, e.g., conjugation to another chemical moiety such as, for example, polyethylene glycol, albumin (e.g., human serum albumin), phosphorylation, and glycosylation.

The term “% sequence identity” is used interchangeably herein with the term “% identity” and refers to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. In certain embodiments, the % identity is selected from, e.g., at least 60%, at least 65%, at least 70%, at least 75%, at least 80%, at least 85%, at least 90%, at least 95%, or at least 99% or more sequence identity to a given sequence. In certain embodiments, the % identity is in the range of, e.g., about 60% to about 70%, about 70% to about 80%, about 80% to about 85%, about 85% to about 90%, about 90% to about 95%, or about 95% to about 99%.

The term “% sequence homology” is used interchangeably herein with the term “% homology” and refers to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In certain embodiments, the % homology is selected from, e.g., at least 60%, at least 65%, at least 70%, at least 75%, at least 80%, at least 85%, at least 90%, at least 95%, or at least 99% or more sequence homology to a given sequence. In certain embodiments, the % homology is in the range of, e.g., about 60% to about 70%, about 70% to about 80%, about 80% to about 85%, about 85% to about 90%, about 90% to about 95%, or about 95% to about 99%.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at the NCBI website. See also Altschul et al., 1990, J. Mol. Biol. 215:403-10 (with special reference to the published default setting, i.e., parameters w=4, t=17) and Altschul et al., 1997, Nucleic Acids Res., 25:3389-3402. Sequence searches are typically carried out using the BLASTP program when evaluating a given amino acid sequence relative to amino acid sequences in the GenBank Protein Sequences and other public databases. The BLASTX program is preferred for searching nucleic acid sequences that have been translated in all reading frames against amino acid sequences in the GenBank Protein Sequences and other public databases. Both BLASTP and BLASTX are run using default parameters of an open gap penalty of 11.0, and an extended gap penalty of 1.0, and utilize the BLOSUM-62 matrix. (Id).

In addition to calculating percent sequence identity, the BLAST algorithm also performs a statistical analysis of the similarity between two sequences (see, e.g., Karlin & Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is, e.g., less than about 0.1, less than about 0.01, or less than about 0.001.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

A protein or polypeptide is “substantially pure,” “substantially homogeneous,” or “substantially purified” when at least about 60% to 75% of a sample exhibits a single species of polypeptide. A substantially pure polypeptide or protein will typically comprise about 50%, 60%, 70%, 80% or 90% W/W of a protein sample, more usually about 95%, and e.g., will be over 99% pure. Protein purity or homogeneity may be indicated by a number of means well known in the art, such as polyacrylamide gel electrophoresis of a protein sample, followed by visualizing a single polypeptide band upon staining the gel with a stain well known in the art. For certain purposes, higher resolution may be provided by using HPLC or other means well known in the art for purification.

An “antigen binding and antagonizing protein” is a protein comprising a portion that binds to an antigen and, optionally, a scaffold or framework portion that allows the antigen binding portion to adopt a conformation that promotes binding of the isolated antagonistic antigen binding protein to the antigen. Examples of antigen binding and antagonizing proteins include antibodies, antibody fragments (e.g., an antigen binding portion of an antibody), antibody derivatives, and antibody analogs. The isolated antagonistic antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the isolated antagonistic antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129 (2003); Roque et al., Biotechnol. Prog. 20:639-654 (2004). In addition, peptide antibody mimetics (“PAMs”) can be used, as well as scaffolds based on antibody mimetics utilizing fibronection components as a scaffold.

An isolated antagonistic antigen binding protein can have, for example, the structure of a naturally occurring immunoglobulin. An “immunoglobulin” is a tetrameric molecule. In a naturally occurring immunoglobulin, each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N. Y. (1989)) (incorporated by reference in its entirety for all purposes). The variable regions of each light/heavy chain pair form the antibody binding site such that an intact immunoglobulin has two binding sites.

An “antibody” refers to a protein comprising one or more polypeptides substantially or partially encoded by immunoglobulin genes or fragments of immunoglobulin genes and having specificity to a tumor antigen or specificity to a molecule overexpressed in a pathological state. The recognized immunoglobulin genes include the kappa, lambda, alpha, gamma, delta, epsilon and mu constant region genes, as well as subtypes of these genes and myriad of immunoglobulin variable region genes. Light chains (LC) are classified as either kappa or lambda. Heavy chains (HC) are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition.

In a full-length antibody, each heavy chain is comprised of a heavy chain variable region (abbreviated herein as HCVR or V_{H}) and a heavy chain constant region. The heavy chain constant region is comprised of three domains, C_{H1}, C_{H2} and C_{H3} (and in some instances, C_{H4}). Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR₁, CDR₁, FR₂, CDR₂, FR₃, CDR₃, FR₄. The extent of the framework region and CDRs has been defined. The sequences of the framework regions of different light or heavy chains are relatively conserved within a species, such as humans. The framework region of an antibody, that is the combined framework regions of the constituent light and heavy chains, serves to position and align the CDRs in three-dimensional space. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

Antibodies exist as intact immunoglobulins or as a number of well characterized fragments. Such fragments include Fab fragments, Fab′ fragments, Fab₂, F(ab)′₂fragments, single chain Fv proteins (“scFv”) and disulfide stabilized Fv proteins (“dsFv”), that bind to the target antigen. A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. While various antibody fragments are defined in terms of the digestion of an intact antibody, one of skill will appreciate that such fragments may be synthesized de novo either chemically or by utilizing recombinant DNA methodology. Thus, as used herein, the term antibody encompasses e.g., monoclonal antibodies (including full-length monoclonal antibodies), polyclonal antibodies, multispecific antibodies (e.g., bispecific antibodies) formed from at least two intact antibodies, human antibodies, humanized antibodies, camelised antibodies, chimeric antibodies, single-chain Fvs (scFv), single-chain antibodies, single domain antibodies, domain antibodies, Fab fragments, F(ab′)₂ fragments, antibody fragments that exhibit the desired biological activity, disulfide-linked Fvs (sdFv), intrabodies, and epitope-binding fragments or antigen binding fragments of any of the above.

A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the V_{H} and C.sub.H1 domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634, 6,696,245, US App. Pub. No. 05/0202512, 04/0202995, 04/0038291, 04/0009507, 03/0039958, Ward et al., Nature 341:544-546 (1989)).

A single-chain antibody (scFv) is an antibody in which a V_{L} and a V_{H} region are joined via a linker (e.g., a synthetic sequence of amino acid residues) to form a continuous protein chain wherein the linker is long enough to allow the protein chain to fold back on itself and form a monovalent antigen binding site (see, e.g., Bird et al., Science 242:423-26 (1988) and Huston et al., 1988, Proc. Natl. Acad. Sci. USA 85:5879-83 (1988)). Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48 (1993), and Poljak et al., Structure 2:1121-23 (1994)). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

An isolated antagonistic antigen binding protein may have one or more binding sites. If there is more than one binding site, the binding sites may be identical to one another or may be different. For example, a naturally occurring human immunoglobulin typically has two identical binding sites, while a “bispecific” or “bifunctional” antibody has two different binding sites.

The term “human antibody” includes all antibodies that have one or more variable and constant regions derived from human immunoglobulin sequences. In one embodiment, all of the variable and constant domains are derived from human immunoglobulin sequences (a fully human antibody). These antibodies may be prepared in a variety of ways, examples of which are described below, including through the immunization with an antigen of interest of a mouse that is genetically modified to express antibodies derived from human heavy and/or light chain-encoding genes.

A “humanized antibody” has a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

An isolated antagonistic antigen binding protein of the present disclosure, including an antibody, “specifically binds” to an antigen, such as the human glucagon receptor if it binds to the antigen with a high binding affinity as determined by a dissociation constant (Kd, or corresponding Kb, as defined below) value of 10⁻⁷ M or less. An isolated antagonistic antigen binding protein that specifically binds to the human glucagon receptor may be able to bind to glucagon receptors from other species as well with the same or different affinities.

An “epitope” is the portion of a molecule that is bound by an isolated antagonistic antigen binding protein (e.g., by an antibody). An epitope can comprise non-contiguous portions of the molecule (e.g., in a polypeptide, amino acid residues that are not contiguous in the polypeptide's primary sequence but that, in the context of the polypeptide's tertiary and quaternary structure, are near enough to each other to be bound by an antigen binding and antagonizing protein).

The term “blood glucose level”, or “level of blood glucose” shall mean blood glucose concentration. In certain embodiments, a blood glucose level is a plasma glucose level. Plasma glucose may be determined in accordance with, e.g., Etgen et al., Metabolism, 49(5): 684-688, 2000) or calculated from a conversion of whole blood glucose concentration in accordance with D'Orazio et al., Clin. Chem. Lab. Med., 44(12):1486-1490, 2006.

The term “normal glucose levels” refers to mean plasma glucose values in humans of less than about 100 mg/dL for fasting levels, and less than about 145 mg/dL for 2-hour post-prandial levels or 125 mg/dL for a random glucose. The term “elevated blood glucose level” or “elevated levels of blood glucose” shall mean an elevated blood glucose level such as that found in a subject demonstrating clinically inappropriate basal and postprandial hyperglycemia or such as that found in a subject in oral glucose tolerance test (oGTT), with “elevated levels of blood glucose” being greater than about 100 mg/dL when tested under fasting conditions, and greater than about 200 mg/dL when tested at 1 hour.

The terms “glucagon inhibitor”, “glucagon suppressor” and “glucagon antagonist” are used interchangeably. Each is a molecule that detectably inhibits glucagon signaling. The inhibition caused by an inhibitor need not be complete so long as the inhibition is detectable using an assay that is recognized and understood in the art as being determinative of glucagon signaling inhibition.

A “pharmaceutical composition” refers to a composition suitable for pharmaceutical use in an animal or human. A pharmaceutical composition comprises a pharmacologically and/or therapeutically effective amount of an active agent and a pharmaceutically acceptable carrier. “Pharmaceutically acceptable carrier” refers to compositions that do not produce adverse, allergic, or other untoward reactions when administered to an animal or a human. As used herein “pharmaceutically acceptable carrier” refers to any of the standard pharmaceutical carriers, vehicles, buffers, and carriers, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

As used herein, a “therapeutically effective amount” of an isolated antagonistic antigen binding protein that specifically binds the human glucagon receptor refers to an amount of such protein that, when provided to a subject in accordance with the disclosed and claimed methods effects one of the following biological activities: treats heart failure; or reduces, suppresses, attenuates, or inhibits one or more symptoms of heart failure.

The terms “treat”, “treating” and “treatment” refer refers to an approach for obtaining beneficial or desired clinical results. The terms “treat”, “treating” and “treatment” refer to a method of alleviating or ameliorating a biological disorder and/or at least one of its attendant symptoms. As used herein, to “manage” or “ameliorate” a disease, disorder or condition means reducing the severity and/or occurrence frequency of the symptoms of the disease, disorder, or condition. Further, references herein to “treatment” include references to curative, palliative and prophylactic treatment. For purposes of this disclosure, beneficial or desired clinical results include, but are not limited to, one or more of the following: improvement in blood glucose to within about 80-180 mg/dL, or to within about 80-170 mg/dL, or to within about 80-160 mg/dL, or to within about 80-150 mg/dL, or to within about 80-140 mg/dL, or an improvement in any one or more conditions, diseases, or symptoms associated with, or resulting from, elevated levels of blood glucose including, but not limited to, hyperglycemia, hyperglucanemia, and hyperinsulinemia.

As used herein and in the appended claims, the singular forms “a,” “or,” and “the” include plural referents unless the context clearly dictates otherwise. It is understood that aspects and variations of the disclosure described herein include “consisting” and/or “consisting essentially of” aspects and variation.

Reference to “about” a value or parameter herein includes (and describes) variations that are directed to that value or parameter per se. For example, description referring to “about X” includes description of “X”.

### Rare Genetic Disorders

There are a number of extremely rare genetic disorders which are characterized, in part, by abnormal resistance to insulin and/or hyperglycemia. Such disorders include, e.g., Maturity Onset Diabetes of the Young Syndrome (MODY)(see, e.g., US20160213625; US20160244525), Wolfram Syndrome (see, e.g., US20150246037), Neonatal Diabetes (see, e.g., US20110112015), Maternally Inherited Diabetes and Deafness Syndrome (see, e.g., US20090143279), Prader-Willi Syndrome (see, e.g., US20160228449), Rabson-Mendenhall Syndrome (see, e.g., US20110178032; US20160115213), Insulin-resistant Acanthosis Nigricans (IRAN) Type A (see, e.g., US20150080345), Leprechaunism (see, e.g., US20110178032), Lipodystrophies (see, e.g., US20150191427), Fanconi Bickel Syndrome (FBS)(see, e.g., US20150273016), Friedreich Ataxia (see, e.g., US20160178610), Laurence-Moon Syndrome (see, e.g., US20080167384), and a class of rare genetic disorders referred to as ‘ciliopathies’, including e.g., Alström syndrome (ALMS)(see, e.g., US20050214757), Bardet-Biedl syndrome (BBS)(see, e.g., US 20030152963), nephronophthisis (NPHP)(see, e.g., US20080044831), Senior-Löken syndrome (SNLS)(see, e.g., US20120210450), Meckel syndrome (MKS)(see, e.g., US20140072975), Joubert syndrome (JBTS)(see, e.g., US20140186833), Oral-facial-digital Type I (OFD 1)(see, e.g., US20120094959), Jeune asphyxiating thoracic dystrophy (JATD), Ellis van Creveld (EVC), Leber congenital amaurosis (LCA)(see, e.g., US20140364488), and polycystic kidney diseases (PKD)(see, e.g., US20150374437).

Maturity Onset Diabetes of the Young Syndrome (MODY) is an autosomal dominant form of diabetes typically occurring before 25 years of age and caused by primary insulin secretion defects. Although MODY is typically used to indicate autosomal dominant noninsulin-dependent diabetes diagnosed before the age of 25 years, there is an increasing incidence of polygenic T2D in childhood and adolescence, and patients with gene mutations characteristic of MODY often present with clinical diabetes later in life (Glaser, Lancet, 361:272-273, 2003). 5 major diagnostic criteria for MODY are usually accepted: (1) hyperglycemia usually diagnosed before age 25 years in at least 1 and ideally 2 family members; (2) autosomal dominant inheritance, with a vertical transmission of diabetes through at least 3 generations, and a similar phenotype shared by diabetic family members; (3) absence of insulin therapy at least 5 years after diagnosis or significant C-peptide levels even in a patient on insulin treatment; (4) insulin levels that are often in the normal range, although inappropriately low for the degree of hyperglycemia, suggesting a primary defect in beta-cell function; and (5) overweight or obesity is rarely associated (and is not required for the development of diabetes) (Vaxillaire et al., Endocr. Rev., 29:254-264, 2008).

Rabson-Mendenhall Syndrome is a rare autosomal recessive disorder characterized by severe insulin resistance. This syndrome usually affects children and has a prognosis of 1-2 years. The disorder is caused by mutations in the insulin receptor gene. Major symptoms of Rabson-Mendenhall syndrome include abnormalities of the teeth and nails, such as dental dysplasia, and deformities of the head and face, which include a coarse prematurely-aged facial appearance with a prominent jaw. A skin abnormality known as acanthosis nigricans, which involves a discoloration (hyperpigmentation) and “velvety” thickening (hyperkeratosis) of the skin around skin fold regions of the neck, groin and under arms is also a common symptom (Parveen et al, International Journal of Dermatology, 47(8): 839-841, 2008). There is no known cure for Rabson-Mendenhall syndrome. Treatment involves controlling blood glucose levels by using insulin and incorporating a strategically planned, controlled diet.

Wolfram Syndrome is a rare and severe autosomal recessive neurodegenerative disease characterized by diabetes mellitus, optic atrophy, diabetes insipidus, and deafness (DIDMOAD). Only insulin-dependent diabetes mellitus and bilateral progressive optic atrophy are necessary to make the diagnosis of DIDMOAD. The first symptom is typically diabetes mellitus, which is usually diagnosed around the age of 6. The next symptom to appear is often optic atrophy, the wasting of optic nerves, around the age of 11. The first signs of optic atrophy are loss of color vision and peripheral vision. The condition worsens over time, and people with optic atrophy are usually blind within 8 years of the first symptoms. Life expectancy of people suffering from this syndrome is about 30 years. There is no known direct treatment. Current treatment efforts focus on managing the complications of Wolfram syndrome, such as diabetes mellitus and diabetes insipidus (Urano, Current Diabetes Reports, 16(1):6, 2016).

Ciliopathies are an emerging class of genetic multi-systemic human disorders that are caused by a multitude of largely unrelated genes that affect ciliary structure/function. They are unified by shared clinical features, such as mental retardation, cystic kidney, retinal defects and polydactyly, and by the common localization of the protein products of these genes at or near the primary cilium of cells. With the realization that many previously disparate conditions are a part of this spectrum of disorders, there has been tremendous interest in the function of cilia in developmental signaling and homeostasis. Ciliopathies are mostly inherited as simple recessive traits, but phenotypic expressivity is under the control of numerous genetic modifiers, putting these conditions at the interface of simple and complex genetics (Lee and Gleeson, Genome Med., 3(9): 59, 2011).

Although many of these syndromes have been recognized as isolated entities, it has only been in the last few years that this seemingly disparate collection of rare and clinically perplexing disorders has been classified. Classification of ciliopathies defines two groups: ciliopathies with skeletal involvement (Jeune asphyxiating thoracic dystrophy (JATD), Oral-facial-digital Type I (OFD 1), Ellis van Creveld (EVC)) and those without (Bardet-Biedl syndrome (BBS), nephronophthisis (NPHP), Alström syndrome (ALMS), Meckel syndrome (MKS), Joubert syndrome (JBTS), Leber congenital amaurosis (LCA)) (Tobin and Beales, Genetics in Medicine, 11:386-402, 2009).

Ciliopathies can affect multiple organs (e.g., Alström syndrome, Bardet-Biedl syndrome or Joubert syndrome) or they might only affect a single organ (e.g., Leber congenital amaurosis or autosomal recessive or dominant polycystic kidney disease). The majority of ciliopathies are rare; the most common is autosomal dominant polycystic kidney disease (ADPKD), which has a population prevalence of ˜0.1%. Research into the ciliopathies is providing fresh insights into the pathogenetic mechanisms of common disorders that share clinical symptoms with the ciliopathies, including obesity, type 2 diabetes mellitus (T2DM), retinal degeneration, hearing loss, cardiomyopathy and renal failure. A thorough review of the structural, metabolic, and developmental defects of the ciliopathies is reported in, e.g., Girald and Petrovsky, Nature Reviews, 7:77-88, 2011.

Alstrom syndrome (ALMS) is a very rare autosomal-recessive disorder caused solely by mutations in ALMS1. Symptoms of ALMS include childhood obesity (95% of patients), type 2 diabetes (T2DM), retinal degeneration, cardiomyopathy, renal impairment, infertility and sensorineuronal hearing loss (Girard and Petrovsky, Nat. Rev. Endocrinol. 7:77-88, 2011). These symptoms lead to high morbidity and reduced life expectancy. Within the first 5 years of life, patients with ALMS develop truncal obesity, insulin resistance, hyperglycemia, hyperleptinemia, hyperinsulinemia, hyperlipidemia, which progress to clinical T2DM and patients with ALMS have a high rate of T2DM (˜70% by 20 years of age). A common clinical characteristic in patients with ALMS is acanthosis nigricans, which is usually a marker of severe insulin resistance. Patients with ALMS often have abnormal liver function secondary to non-alcoholic fatty liver disease. The main causes of death in patients with ALMS are heart, kidney or liver failure. T2DM and loss of vision and hearing are the major causes of morbidity. Mouse models of ALMS also exhibit most of the clinical symptoms of ALMS seen in human patients, including obesity, hyperglycemia, hyperinsulinemia, hyperlipidemia, steatohepatitis, infertility and sensorineuronal hearing loss (Id).

Bardet-Biedl syndrome (BBS) is a rare autosomal recessive ciliopathy characterized by retinal dystrophy, obesity, post-axial polydactyly, renal dysfunction, learning difficulties and hypogonadism (Forsythe and Beales, European Journal of Human Genetics, 21: 8-13, 2013). BBS is caused by mutations in at least 16 genes involved in primary cilium function. Most BBS patients are diagnosed in late childhood or early adulthood and obesity incidence is reported to be 72-86% in the BBS population. The development of type 2 diabetes is prevalent among patients. It may be related to the level of obesity and it is often found in association with other signs of metabolic syndrome. Other clinical symptoms including mild developmental delay, speech delay, poor coordination, and an increased incidence of hypertension and congenital cardiovascular disease have been reported in BBS patients (Id). Renal abnormalities can be major cause of morbidity and mortality in BBS (O'Dea et al., Am J Kidney Dis, 27:776-783, 1997).

Neonatal Diabetes (NDM) is an insulin-requiring hyperglycemia form of diabetes that occurs in the first 3 months of life. It is a rare disease, occurring in only one in 100,000 to 400,000 live births (Shield, J. P. H., Horm. Res. 53(1): 7-11, 2000). Infants with NDM do not produce enough insulin, leading to an increase in blood glucose. NDM can be mistaken for the much more common type 1 diabetes, but type 1 diabetes usually occurs later than the first 6 months of life. In about half of those with NDM, the condition is lifelong and is called permanent neonatal diabetes mellitus (PNDM). In the rest of those with NDM, the condition is transient and disappears during infancy but can reappear later in life; this type of NDM is called transient neonatal diabetes mellitus (TNDM). Specific genes that can cause NDM have been identified Symptoms of NDM include frequent urination, and dehydration. NDM can be diagnosed by finding elevated levels of glucose in blood or urine. In severe cases, the deficiency of insulin may cause the body to produce an excess of acid, resulting in a potentially life-threatening condition called ketoacidosis. Most fetuses with NDM do not grow well in the womb and newborns are much smaller than those of the same gestational age, a condition called intrauterine growth restriction. After birth, some infants fail to gain weight and growth as rapidly as other infants of the same age and sex. Appropriate therapy improves and may normalize growth and development

Prader-Willi Syndrome is a genetic disorder due to loss of function of specific genes on chromosome 15. In newborns symptoms include weak muscles, poor feeding, and slow development. In childhood the person becomes constantly hungry which often leads to obesity and type 2 diabetes. There is also typically mild to moderate intellectual impairment and behavioral problems. Often the forehead is narrow, hands and feet small, height short, skin light in color, and they are unable to have children. Given the complexity of PWS, its symptoms are treated with a number of different medications, including human growth hormone (GH), psychiatric/mood stabilizing medications, and wakefulness promoting agents. Many of the aforementioned drugs that are currently used to treat PWS have undesirable side effects. Accordingly, there is a need in the art to reduce these side effects by providing a way to reduce the dosages of the drugs used to treat PWS, or eliminate the need to use such drugs.

### Glucagon Receptor and Antigen Binding and Antagonizing Proteins

Glucagon is a 29 amino acid hormone processed from its pre-pro-form in the pancreatic alpha cells by cell specific expression of prohormone convertase 2 (PC2), a neuroendocrine-specific protease involved in the intracellular maturation of prohormones and proneuropeptides (Furuta et al., J. Biol. Chem. 276: 27197-27202 (2001)). In vivo, glucagon is a major counter-regulatory hormone for insulin actions. During fasting, glucagon secretion increases in response to falling glucose levels. Increased glucagon secretion stimulates glucose production by promoting hepatic glycogenolysis and gluconeogenesis (Dunning and Gerich, Endocrine Reviews, 28:253-283 (2007)). Thus glucagon counterbalances the effects of insulin in maintaining normal levels of glucose in animals.

The biological effects of glucagon are mediated through the binding and subsequent activation of a specific cell surface receptor, the glucagon receptor. The glucagon receptor (GCGR) is a member of the secretin subfamily (family B) of G-protein-coupled receptors. The human GCGR is a 477 amino acid sequence GPCR and the amino acid sequence of GCGR is highly conserved across species (Mayo et al, Pharmacological Rev., 55:167-194, (2003)). The glucagon receptor is predominantly expressed in the liver, where it regulates hepatic glucose output, on the kidney, and on islet β-cells, reflecting its role in gluconeogenesis. The activation of the glucagon receptors in the liver stimulates the activity of adenyl cyclase and phosphoinositol turnover which subsequently results in increased expression of gluconeogenic enzymes including phosphoenolpyruvate carboxykinase (PEPCK), fructose-1,6-bisphosphatase (FBPase-1), and glucose-6-phosphatase (G-6-Pase). In addition, glucagon signaling activates glycogen phosphorylase and inhibits glycogen synthase. Studies have shown that higher basal glucagon levels and lack of suppression of postprandial glucagon secretion contribute to diabetic conditions in humans (Muller et al., N Eng J Med 283: 109-115 (1970)). As such, methods of controlling and lowering blood glucose by targeting glucagon production or function using a GCGR antagonist have been explored.

In various embodiments, the antigen binding and antagonizing proteins of the present disclosure may be selected to bind to membrane-bound glucagon receptors as expressed on cells, and inhibit or block glucagon signaling through the glucagon receptor. In various embodiments, the antigen binding and antagonizing proteins of the present disclosure specifically bind to the human glucagon receptor. In various embodiments, the antigen binding and antagonizing proteins binding to the human glucagon receptor may also bind to the glucagon receptors of other species. The polynucleotide and polypeptide sequences for several species of glucagon receptor are known (see, e.g., U.S. Pat. No. 7,947,809, herein incorporated by reference in its entirety for its specific teaching of polynucleotide and polypeptide sequences of a human, rat, mouse and cynomolgus glucagon receptor). In various embodiments of the present disclosure, the antigen binding and antagonizing proteins specifically bind the human glucagon receptor having the amino acid sequence set forth in SEQ ID NO: 1:

Glucagon Receptor Human (Homo sapiens) amino acid
sequence (Accession Number AAI04855)
(SEQ ID NO: 1)
MPPCQPQRPLLLLLLLLACQPQVPSAQVMDFLFEKWKLYGDQCHHNLSLL
PPPTELVCNRTFDKYSCWPDTPANTTANISCPWYLPWHHKVQHRFVFKRC
GPDGQWVRGPRGQPWRDASQCQMDGEEIEVQKEVAKMYSSFQVMYTVGYS
LSLGALLLALAILGGLSKLHCTRNAIHANLFASFVLKASSVLVIDGLLRT
RYSQKIGDDLSVSTWLSDGAVAGCRVAAVFMQYGIVANYCWLLVEGLYLH
NLLGLATLPERSFFSLYLGIGWGAPMLFVVPWAVVKCLFENVQCWTSNDN
MGFWWILRFPVFLAILINFFIFVRIVQLLVAKLRARQMHHTDYKFRLAKS
TLTLIPLLGVHEVVFAFVTDEHAQGTLRSAKLFFDLFLSSFQGLLVAVLY
CFLNKEVQSELRRRWHRWRLGKVLWEERNTSNHRASSSPGHGPPSKELQF
GRGGGSQDSSAETPLAGGLPRLAESPF

In various embodiments, the antigen binding and antagonizing proteins of the present disclosure specifically bind glucagon receptors which have at least 70%, at least 75%, at least 80%, at least 85%, at least 90%, at least 95%, at least 96%, at least 97%, at least 98%, or at least 99% identity (as calculated using methods known in the art and described herein) to the glucagon receptors described in the cited references are also included in the present disclosure.

The antigen binding and antagonizing proteins of the present disclosure function to block the interaction between glucagon and its receptor, thereby inhibiting the glucose elevating effects of glucagon. As such, the use of the antigen binding and antagonizing proteins of the present disclosure are an effective means of achieving normal levels of glucose, thereby ameliorating, or preventing one or more symptoms of, or long term complications caused by diabetes including, but not limited to, hyperglycemia, hyperglucanemia, and hyperinsulinemia.

Methods of generating antibodies that bind to antigens such as the human glucagon receptor are known to those skilled in the art. For example, a method for generating a monoclonal antibody that binds specifically to a targeted antigen polypeptide may comprise administering to a mouse an amount of an immunogenic composition comprising the targeted antigen polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the targeted antigen polypeptide. Once obtained, a hybridoma can be propagated in a cell culture, optionally in culture conditions where the hybridoma-derived cells produce the monoclonal antibody that binds specifically to targeted antigen polypeptide. The monoclonal antibody may be purified from the cell culture. A variety of different techniques are then available for testing an antigen/antibody interaction to identify particularly desirable antibodies.

Other suitable methods of producing or isolating antibodies of the requisite specificity can used, including, for example, methods which select recombinant antibody from a library, or which rely upon immunization of transgenic animals (e.g., mice) capable of producing a full repertoire of human antibodies. See e.g., Jakobovits et al., Proc. Natl. Acad. Sci. (U.S.A.), 90: 2551-2555, 1993; Jakobovits et al., Nature, 362: 255-258, 1993; Lonberg et al., U.S. Pat. No. 5,545,806; and Surani et al., U.S. Pat. No. 5,545,807.

Antibodies can be engineered in numerous ways. They can be made as single-chain antibodies (including small modular immunopharmaceuticals or SMIPs™), Fab and F(ab′)2 fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

Chimeric antibodies can be produced by recombinant DNA techniques known in the art. For example, a gene encoding the Fc constant region of a murine (or other species) monoclonal antibody molecule is digested with restriction enzymes to remove the region encoding the murine Fc, and the equivalent portion of a gene encoding a human Fc constant region is substituted (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science, 240:1041-1043, 1988; Liu et al., Proc. Natl. Acad. Sci. (U.S.A.), 84:3439-3443, 1987; Liu et al., J. Immunol., 139:3521-3526, 1987; Sun et al., Proc. Natl. Acad. Sci. (U.S.A.), 84:214-218, 1987; Nishimura et al., Canc. Res., 47:999-1005, 1987; Wood et al., Nature, 314:446-449, 1985; and Shaw et al., J. Natl Cancer Inst., 80:1553-1559, 1988).

Methods for humanizing antibodies have been described in the art. In some embodiments, a humanized antibody has one or more amino acid residues introduced from a source that is nonhuman, in addition to the nonhuman CDRs. Humanization can be essentially performed following the method of Winter and co-workers (Jones et al., Nature, 321:522-525, 1986; Riechmann et al., Nature, 332:323-327, 1988; Verhoeyen et al., Science, 239:1534-1536, 1988), by substituting hypervariable region sequences for the corresponding sequences of a human antibody. Accordingly, such “humanized” antibodies are chimeric antibodies (U.S. Pat. No. 4,816,567) wherein substantially less than an intact human variable region has been substituted by the corresponding sequence from a nonhuman species. In practice, humanized antibodies are typically human antibodies in which some hypervariable region residues and possibly some framework region residues are substituted by residues from analogous sites in rodent antibodies.

U.S. Pat. No. 5,693,761 to Queen et al, discloses a refinement on Winter et al. for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable region sequences are compared to a particular mouse sequence and the percentage identity between correspondent framework residues is calculated. The human variable region with the highest percentage is selected to provide the framework sequences for the humanizing project. Queen also teaches that it is important to retain in the humanized framework, certain amino acid residues from the mouse framework critical for supporting the CDRs in a binding-capable conformation. Potential criticality is assessed from molecular models. Candidate residues for retention are typically those adjacent in linear sequence to a CDR or physically within 6Å of any CDR residue.

In other approaches, the importance of particular framework amino acid residues is determined experimentally once a low-avidity humanized construct is obtained, by reversion of single residues to the mouse sequence and assaying antigen binding as described by Riechmann et al, 1988. Another example approach for identifying important amino acids in framework sequences is disclosed by U.S. Pat. No. 5,821,337 to Carter et al, and by U.S. Pat. No. 5,859,205 to Adair et al. These references disclose specific Kabat residue positions in the framework, which, in a humanized antibody may require substitution with the correspondent mouse amino acid to preserve avidity.

Another method of humanizing antibodies, referred to as “framework shuffling”, relies on generating a combinatorial library with nonhuman CDR variable regions fused in frame into a pool of individual human germline frameworks (Dall'Acqua et al., Methods, 36:43, 2005). The libraries are then screened to identify clones that encode humanized antibodies which retain good binding.

The choice of human variable regions, both light and heavy, to be used in making the humanized antibodies is very important to reduce antigenicity. According to the so-called “best-fit” method, the sequence of the variable region of a rodent antibody is screened against the entire library of known human variable-domain sequences. The human sequence that is closest to that of the rodent is then accepted as the human framework region (framework region) for the humanized antibody (Sims et al., J. Immunol., 151:2296, 1993; Chothia et al., J. Mol. Biol., 196:901, 1987). Another method uses a particular framework region derived from the consensus sequence of all human antibodies of a particular subgroup of light or heavy chain variable regions. The same framework may be used for several different humanized antibodies (Carter et al., Proc. Natl. Acad. Sci. (U.S.A.), 89:4285, 1992; Presta et al., J. Immunol., 151:2623, 1993).

The choice of nonhuman residues to substitute into the human variable region can be influenced by a variety of factors. These factors include, for example, the rarity of the amino acid in a particular position, the probability of interaction with either the CDRs or the antigen, and the probability of participating in the interface between the light and heavy chain variable domain interface. (See, for example, U.S. Pat. Nos. 5,693,761, 6,632,927, and 6,639,055). One method to analyze these factors is through the use of three-dimensional models of the nonhuman and humanized sequences. Three-dimensional immunoglobulin models are commonly available and are familiar to those skilled in the art. Computer programs are available that illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind its antigen. In this way, nonhuman residues can be selected and substituted for human variable region residues in order to achieve the desired antibody characteristic, such as increased affinity for the target antigen(s).

Methods for making fully human antibodies have been described in the art. By way of example, a method for producing an anti-GCGR antibody or antigen binding antibody fragment thereof comprises the steps of synthesizing a library of human antibodies on phage, screening the library with GCGR or an antibody binding portion thereof, isolating phage that bind GCGR, and obtaining the antibody from the phage. By way of another example, one method for preparing the library of antibodies for use in phage display techniques comprises the steps of immunizing a non-human animal comprising human immunoglobulin loci with GCGR or an antigenic portion thereof to create an immune response, extracting antibody-producing cells from the immunized animal; isolating RNA encoding heavy and light chains of antibodies of the disclosure from the extracted cells, reverse transcribing the RNA to produce cDNA, amplifying the cDNA using primers, and inserting the cDNA into a phage display vector such that antibodies are expressed on the phage. Recombinant anti-GCGR antibodies of the disclosure may be obtained in this way.

Again, by way of example, recombinant human anti-GCGR antibodies of the disclosure can also be isolated by screening a recombinant combinatorial antibody library. Preferably the library is a scFv phage display library, generated using human V_{L} and V_{H} cDNAs prepared from mRNA isolated from B cells. Methods for preparing and screening such libraries are known in the art. Kits for generating phage display libraries are commercially available (e.g., the Pharmacia Recombinant Phage Antibody System, catalog no. 27-9400-01; and the Stratagene SurfZAP™ phage display kit, catalog no. 240612). There also are other methods and reagents that can be used in generating and screening antibody display libraries (see, e.g., U.S. Pat. No. 5,223,409; PCT Publication Nos. WO 92/18619, WO 91/17271, WO 92/20791, WO 92/15679, WO 93/01288, WO 92/01047, WO 92/09690; Fuchs et al., Bio/Technology, 9:1370-1372 (1991); Hay et al., Hum. Antibod. Hybridomas, 3:81-85, 1992; Huse et al., Science, 246:1275-1281, 1989; McCafferty et al., Nature, 348:552-554, 1990; Griffiths et al., EMBO J., 12:725-734, 1993; Hawkins et al., J. Mol. Biol., 226:889-896, 1992; Clackson et al., Nature, 352:624-628, 1991; Gram et al., Proc. Natl. Acad. Sci. (U.S.A.), 89:3576-3580, 1992; Garrad et al., Bio/Technology, 9:1373-1377, 1991; Hoogenboom et al., Nuc. Acid Res., 19:4133-4137, 1991; and Barbas et al., Proc. Natl. Acad. Sci. (U.S.A.), 88:7978-7982, 1991), all incorporated herein by reference.

Human antibodies are also produced by immunizing a non-human, transgenic animal comprising within its genome some or all of human immunoglobulin heavy chain and light chain loci with a human IgE antigen, e.g., a XenoMouse™ animal (Abgenix, Inc./Amgen, Inc.—Fremont, Calif.). XenoMouse™ mice are engineered mouse strains that comprise large fragments of human immunoglobulin heavy chain and light chain loci and are deficient in mouse antibody production. See, e.g., Green et al., Nature Genetics, 7:13-21, 1994 and U.S. Pat. Nos. 5,916,771, 5,939,598, 5,985,615, 5,998,209, 6,075,181, 6,091,001, 6,114,598, 6,130,364, 6,162,963 and 6,150,584. XenoMouse™ mice produce an adult-like human repertoire of fully human antibodies and generate antigen-specific human antibodies. In some embodiments, the XenoMouse™ mice contain approximately 80% of the human antibody V gene repertoire through introduction of megabase sized, germline configuration fragments of the human heavy chain loci and kappa light chain loci in yeast artificial chromosome (YAC). In other embodiments, XenoMouse™ mice further contain approximately all of the human lambda light chain locus. See Mendez et al., Nature Genetics, 15:146-156, 1997; Green and Jakobovits, J. Exp. Med., 188:483-495, 1998; and WO 98/24893.

In various embodiments, the isolated antagonistic antigen binding protein of the present disclosure utilize an antibody or antigen binding antibody fragment thereof is a polyclonal antibody, a monoclonal antibody or antigen-binding fragment thereof, a recombinant antibody, a diabody, a chimerized or chimeric antibody or antigen-binding fragment thereof, a humanized antibody or antigen-binding fragment thereof, a fully human antibody or antigen-binding fragment thereof, a CDR-grafted antibody or antigen-binding fragment thereof, a single chain antibody, an Fv, an Fd, an Fab, an Fab′, or an F(ab′)₂, and synthetic or semi-synthetic antibodies.

In various embodiments, the isolated antagonistic antigen binding protein of the present disclosure utilize an antibody or antigen-binding fragment that binds to an immune-checkpoint protein antigen with a dissociation constant (K_{D}) of, e.g., at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M. In various embodiments, the isolated antagonistic antigen binding protein of the present disclosure utilize an antibody or antigen-binding fragment that binds to an immune-checkpoint protein antigen with a dissociation constant (K_{D}) in the range of, e.g., at least about 1×10⁻⁷ M to at least about 1×10⁻⁸ M, at least about 1×10⁻⁸ M to at least about 1×10⁻⁹ M, at least about 1×10⁻⁹ M to at least about 1×10⁻¹⁰ M, at least about 1×10⁻¹⁰ M to at least about 1×10⁻¹¹ M, or at least about 1×10⁻¹¹ M to at least about 1×10⁻¹² M.

Antibodies to the glucagon receptor have been described in, e.g., U.S. Pat. Nos. 5,770,445, 7,947,809, 7,968,686, 8,545,847, and 8,771,696; European patent application EP2074149A2; EP patent EP0658200B1; U.S. patent publications 2009/0041784; 2009/0252727; 2013/0344538 and 2014/0335091; and PCT publication WO2008/036341. In various embodiments of the present invention, the isolated antagonistic antigen binding protein is an anti-GCGR (“antagonistic”) antibody or antigen-binding fragment which comprises the polynucleotide and polypeptide sequences set forth in, e.g., U.S. Pat. No. 7,947,809, and 8,158,759, each herein incorporated by reference in its entirety for its specific teaching of polynucleotide and polypeptide sequences of various anti-GCGR antibodies or antigen-binding fragments.

In various embodiments of the present disclosure the antibody may be an anti-GCGR antibody that has the same or higher antigen-binding affinity as that of the antibody comprising the heavy chain variable region sequence as set forth in SEQ ID NO: 2. In various embodiments, the antibody may be an anti-GCGR antibody which binds to the same epitope as the antibody comprising the heavy chain variable region sequence as set forth in SEQ ID NO: 2. In various embodiments, the antibody is an anti-GCGR antibody which competes with the antibody comprising the heavy chain variable region sequence as set forth in SEQ ID NO: 2. In various embodiments, the antibody may be an anti-GCGR antibody which comprises at least one (such as two or three) CDRs of the heavy chain variable region sequence as set forth in SEQ ID NO: 2. In various embodiments, the antibody may be an anti-GCGR antibody which comprises the heavy chain variable region sequence as set forth in SEQ ID NO: 2. In various embodiments, the antibody may be an anti-GCGR antibody which comprises the heavy chain variable region sequence as set forth in SEQ ID NO: 2:

(SEQ ID NO: 2)
QVQLVESGGGVVQPGRSLRLSCAASGFTFSSYGMHWVRQAPGKGLEWVAV
MWYDGSNKDYVDSVKGRFTISRDNSKNTLYLQMNRLRAEDTAVYYCAREK
DHYDILTGYNYYYGLDVWGQGTTVTVSS

In various embodiments of the present disclosure the antibody may be an anti-GCGR antibody that has the same or higher antigen-binding affinity as that of the antibody comprising the light chain variable region sequence as set forth in SEQ ID NO: 3. In various embodiments, the antibody may be an anti-GCGR antibody which binds to the same epitope as the antibody comprising the light chain variable region sequence as set forth in SEQ ID NO: 3. In various embodiments, the antibody is an anti-GCGR antibody which competes with the antibody comprising the light chain variable region sequence as set forth in SEQ ID NO: 3. In various embodiments, the antibody may be an anti-GCGR antibody which comprises at least one (such as two or three) CDRs of the light chain variable region sequence as set forth in SEQ ID NO: 3. In various embodiments, the antibody may be an anti-GCGR antibody which comprises the light chain variable region sequence as set forth in SEQ ID NO: 3. In various embodiments, the antibody may be an anti-GCGR antibody which comprises the light chain variable region sequence as set forth in SEQ ID NO: 3:

(SEQ ID NO: 3)
DIQMTQSPSSLSASVGDRVTITCRASQGIRNDLGWYQQKPGKAPKRLIYAA
SSLQSGVPSRFSGSGSGTEFTLTISSVQPEDFVTYYCLQHNSNPLTFGGGT
KVEIK

In various embodiments, the antibody contains an amino acid sequence that shares an observed homology of, e.g., at least 70%, at least 75%, at least 80%, at least 85%, at least 90%, at least 95%, at least 96%, at least 97%, at least 98%, or at least 99% with the sequences of SEQ ID NOS: 2 or 3.

In various embodiments of the present disclosure the antibody may be an anti-GCGR antibody that has the same or higher antigen-binding affinity as that of the antibody comprising the heavy chain variable region sequence as set forth in SEQ ID NO: 4. In various embodiments, the antibody may be an anti-GCGR antibody which binds to the same epitope as the antibody comprising the heavy chain variable region sequence as set forth in SEQ ID NO: 4. In various embodiments, the antibody is an anti-GCGR antibody which competes with the antibody comprising the heavy chain variable region sequence as set forth in SEQ ID NO: 4. In various embodiments, the antibody may be an anti-GCGR antibody which comprises at least one (such as two or three) CDRs of the heavy chain variable region sequence as set forth in SEQ ID NO: 4. In various embodiments, the antibody may be an anti-GCGR antibody which comprises the heavy chain variable region sequence as set forth in SEQ ID NO: 4. In various embodiments, the antibody may be an anti-GCGR antibody which comprises the heavy chain variable region sequence as set forth in SEQ ID NO: 4:

(SEQ ID NO: 4)
QVQLVESGGGVVQPGRSLRLSCAASGFTFSSYGMHWVRQAPGKGLEWV
AVMWYDGSNKDYVDSVKGRFTISRDNSKNTLYLQMNSLRAEDTAVYYCAR
EKDHYDILTGYNYYYGLDVWGQGTTVTVSS

In various embodiments of the present disclosure the antibody may be an anti-GCGR antibody that has the same or higher antigen-binding affinity as that of the antibody comprising the light chain variable region sequence as set forth in SEQ ID NO: 5. In various embodiments, the antibody may be an anti-GCGR antibody which binds to the same epitope as the antibody comprising the light chain variable region sequence as set forth in SEQ ID NO: 5. In various embodiments, the antibody is an anti-GCGR antibody which competes with the antibody comprising the light chain variable region sequence as set forth in SEQ ID NO: 5. In various embodiments, the antibody may be an anti-GCGR antibody which comprises at least one (such as two or three) CDRs of the light chain variable region sequence as set forth in SEQ ID NO: 5. In various embodiments, the antibody may be an anti-GCGR antibody which comprises the light chain variable region sequence as set forth in SEQ ID NO: 5. In various embodiments, the antibody may be an anti-GCGR antibody which comprises the light chain variable region sequence as set forth in SEQ ID NO: 5:

(SEQ ID NO: 5)
DIQMTQSPSSLSASVGDRVTITCRASQGIRNDLGWYQQKPGKAPKRLIYA
ASSLQSGVPSRFSGSGSGTEFTLTISSLQPEDFVTYYCLQHNSNPLTFGG
GTKVEIK

In various embodiments, the antibody contains an amino acid sequence that shares an observed homology of, e.g., at least 70%, at least 75%, at least 80%, at least 85%, at least 90%, at least 95%, at least 96%, at least 97%, at least 98%, or at least 99% with the sequences of SEQ ID NOS: 4 or 5.

In various embodiments of the present disclosure the antibody may be an anti-GCGR antibody that has the same or higher antigen-binding affinity as that of the antibody comprising the heavy chain variable region sequence as set forth in SEQ ID NO: 6. In various embodiments, the antibody may be an anti-GCGR antibody which binds to the same epitope as the antibody comprising the heavy chain variable region sequence as set forth in SEQ ID NO: 6. In various embodiments, the antibody is an anti-GCGR antibody which competes with the antibody comprising the heavy chain variable region sequence as set forth in SEQ ID NO: 6. In various embodiments, the antibody may be an anti-GCGR antibody which comprises at least one (such as two or three) CDRs of the heavy chain variable region sequence as set forth in SEQ ID NO: 6. In various embodiments, the antibody may be an anti-GCGR antibody which comprises the heavy chain variable region sequence as set forth in SEQ ID NO: 6. In various embodiments, the antibody may be an anti-GCGR antibody which comprises the heavy chain variable region sequence as set forth in SEQ ID NO: 6:

(SEQ ID NO: 6)
QVQLVESGGGVVQPGRSLRLSCAASGFTFSSYGMHWVRQAPGKGLEWVA
VMWYDGSNKDYVDSVKGRFTISRDNSKNTLYLQMNRLRAEDTAVYYCARE
KDHYDILTGYNYYYGLDVWGQGTTVTVSS

In various embodiments of the present disclosure the antibody may be an anti-GCGR antibody that has the same or higher antigen-binding affinity as that of the antibody comprising the light chain variable region sequence as set forth in SEQ ID NO: 7. In various embodiments, the antibody may be an anti-GCGR antibody which binds to the same epitope as the antibody comprising the light chain variable region sequence as set forth in SEQ ID NO: 7. In various embodiments, the antibody is an anti-GCGR antibody which competes with the antibody comprising the light chain variable region sequence as set forth in SEQ ID NO: 7. In various embodiments, the antibody may be an anti-GCGR antibody which comprises at least one (such as two or three) CDRs of the light chain variable region sequence as set forth in SEQ ID NO: 7. In various embodiments, the antibody may be an anti-GCGR antibody which comprises the light chain variable region sequence as set forth in SEQ ID NO: 7. In various embodiments, the antibody may be an anti-GCGR antibody which comprises the light chain variable region sequence as set forth in SEQ ID NO: 7:

(SEQ ID NO: 7)
DIQMTQSPSSLSASVGDRVTITCRASQGIRNDLGWYQQKPGKAPKRLIYA
ASSLESGVPSRFSGSGSGTEFTLTISSVQPEDFVTYYCLQHNSNPLTFGG
GTKVEIK

In various embodiments, the antibody contains an amino acid sequence that shares an observed homology of, e.g., at least 70%, at least 75%, at least 80%, at least 85%, at least 90%, at least 95%, at least 96%, at least 97%, at least 98%, or at least 99% with the sequences of SEQ ID NOS: 6 or 7.

In various embodiments of the present disclosure the antibody may be an anti-GCGR antibody that has the same or higher antigen-binding affinity as that of the chimeric antibody comprising the heavy chain sequence as set forth in SEQ ID NO: 8. In various embodiments, the antibody may be an anti-GCGR antibody which binds to the same epitope as the antibody comprising the heavy chain sequence as set forth in SEQ ID NO: 8. In various embodiments, the antibody is an anti-GCGR antibody which competes with the antibody comprising the heavy chain sequence as set forth in SEQ ID NO: 8. In various embodiments, the antibody may be an anti-GCGR antibody which comprises at least one (such as two or three) CDRs of the heavy chain sequence as set forth in SEQ ID NO: 8. In various embodiments, the antibody may be an anti-GCGR antibody which comprises the heavy chain sequence as set forth in SEQ ID NO: 8. In various embodiments, the antibody may be an anti-GCGR antibody which comprises the heavy chain sequence as set forth in SEQ ID NO: 8:

(SEQ ID NO: 8)
MEFGLSWVFLVALLRGVQCQVQLVESGGGVVQPGRSLRLSCAASGFTFSS
YGMHWVRQAPGKGLEWVAVMWYDGSNKDYVDSVKGRFTISRDNSKNTLYL
QMNRLRAEDTAVYYCAREKDHYDILTGYNYYYGLDVWGQGTTVTVSSAKT
TPPSVYPLAPGSAAQTNSMVTLGCLVKGYFPEPVTVIWNSGSLSSGVHTF
PAVLQSDLYTLSSSVTVPSSTWPSETVTCNVAHPASSTKVDKKIVPRDCG
CKPCICTVPEVSSVFIFPPKPKDVLTITLTPKVTCVVVDISKDDPEVQFS
WFVDDVEVHTAQTQPREEQFNSTFRSVSELPIMHQDWLNGKEFKCRVNSA
AFPAPIEKTISKTKGRPKAPQVYTIPPPKEQMAKDKVSLTCMITDFFPED
ITVEWQWNGQPAENYKNTQPIMDTDGSYFVYSKLNVQKSNWEAGNTFTCS
VLHEGLHNHHTEKSLSHSPGK

In various embodiments of the present disclosure the antibody may be an anti-GCGR antibody that has the same or higher antigen-binding affinity as that of the chimeric antibody comprising the light chain sequence as set forth in SEQ ID NO: 9. In various embodiments, the antibody may be an anti-GCGR antibody which binds to the same epitope as the antibody comprising the light chain sequence as set forth in SEQ ID NO: 9. In various embodiments, the antibody is an anti-GCGR antibody which competes with the antibody comprising the light chain sequence as set forth in SEQ ID NO: 9. In various embodiments, the antibody may be an anti-GCGR antibody which comprises at least one (such as two or three) CDRs of the light chain sequence as set forth in SEQ ID NO: 9. In various embodiments, the antibody may be an anti-GCGR antibody which comprises the light chain sequence as set forth in SEQ ID NO: 9. In various embodiments, the antibody may be an anti-GCGR antibody which comprises the light chain sequence as set forth in SEQ ID NO: 9:

(SEQ ID NO: 9)
MDMRVPAQLLGLLLLWFPGARCDIQMTQSPSSLSASVGDRVTITCRASQG
IRNDLGWYQQKPGKAPKRLIYAASSLESGVPSRFSGSGSGTEFTLTISSV
QPEDFVTYYCLQHNSNPLTFGGGTKVEIKRADAAPTVSIFPPSSEQLTSG
GASVVCFLNNFYPKDINVKWKIDGSERQNGVLNSWTDQDSKDSTYSMSST
LTLTKDEYERHNSYTCEATHKTSTSPIVKSFNRNEC

In various embodiments, the antibody contains an amino acid sequence that shares an observed homology of, e.g., at least 70%, at least 75%, at least 80%, at least 85%, at least 90%, at least 95%, at least 96%, at least 97%, at least 98%, or at least 99% with the sequences of SEQ ID NOS: 8 or 9.

In various embodiments of the present disclosure the antibody may be an anti-GCGR antibody which comprises a heavy chain variable region sequence selected from SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 and SEQ ID NO: 28, and a light chain variable region sequence selected from SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47. In various embodiments, the antibody contains an amino acid sequence that shares an observed homology of, e.g., at least 70%, at least 75%, at least 80%, at least 85%, at least 90%, at least 95%, at least 96%, at least 97%, at least 98%, or at least 99% with the sequences of SEQ ID NOS: 10-28 or SEQ ID NOS: 29-47. Examples of certain anti-GCGR antibodies contemplated for use are provided in Table 2:

TABLE 2
Examples of Anti-GCGR Antibodies

| HCVR | LCVR |
|---|---|
| SEQ ID NO: 2 | SEQ ID NO: 3 |
| SEQ ID NO: 4 | SEQ ID NO: 5 |
| SEQ ID NO: 6 | SEQ ID NO: 7 |
| SEQ ID NO: 10 | SEQ ID NO: 29 |
| SEQ ID NO: 11 | SEQ ID NO: 30 |
| SEQ ID NO: 12 | SEQ ID NO: 31 |
| SEQ ID NO: 13 | SEQ ID NO: 32 |
| SEQ ID NO: 14 | SEQ ID NO: 33 |
| SEQ ID NO: 15 | SEQ ID NO: 34 |
| SEQ ID NO: 16 | SEQ ID NO: 35 |
| SEQ ID NO: 17 | SEQ ID NO: 36 |
| SEQ ID NO: 18 | SEQ ID NO: 37 |
| SEQ ID NO: 19 | SEQ ID NO: 38 |
| SEQ ID NO: 20 | SEQ ID NO: 39 |
| SEQ ID NO: 21 | SEQ ID NO: 40 |
| SEQ ID NO: 22 | SEQ ID NO: 41 |
| SEQ ID NO: 23 | SEQ ID NO: 42 |
| SEQ ID NO: 24 | SEQ ID NO: 43 |
| SEQ ID NO: 25 | SEQ ID NO: 44 |
| SEQ ID NO: 26 | SEQ ID NO: 45 |
| SEQ ID NO: 27 | SEQ ID NO: 46 |
| SEQ ID NO: 28 | SEQ ID NO: 47 |

An isolated anti-GCGR antibody, antibody fragment, or antibody derivative of the present disclosure can comprise any constant region known in the art. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. In various embodiments, the light or heavy chain constant region is a fragment, derivative, variant, or mutein of a naturally occurring constant region.

Techniques are known for deriving an antibody of a different subclass or isotype from an antibody of interest, i.e., subclass switching. Thus, IgG antibodies may be derived from an IgM antibody, for example, and vice versa. Such techniques allow the preparation of new antibodies that possess the antigen-binding properties of a given antibody (the parent antibody), but also exhibit biological properties associated with an antibody isotype or subclass different from that of the parent antibody. Recombinant DNA techniques may be employed. Cloned DNA encoding particular antibody polypeptides may be employed in such procedures, e.g., DNA encoding the constant domain of an antibody of the desired isotype. See also Lanitto et al., Methods Mol. Biol. 178:303-16 (2002).

In various embodiments, an isolated antigen binding protein of the present disclosure comprises the constant light chain kappa region as set forth in SEQ ID NO: 48, or a fragment thereof. In various embodiments, an isolated antigen binding protein of the present disclosure comprises the constant light chain lambda region as set forth in SEQ ID NO: 49, or a fragment thereof. In various embodiments, an isolated antigen binding protein of the present disclosure comprises a IgG2 heavy chain constant region set forth in SEQ ID NO: 50, or a fragment thereof.

In various embodiments, an isolated antagonistic antigen binding protein of the present disclosure comprises a heavy chain sequence as set forth in SEQ ID NO: 51 and comprises a light chain as set forth in SEQ ID NO: 52.

In various embodiments of the present disclosure, the isolated antagonistic antigen binding protein is a hemibody. A “hemibody” is an immunologically-functional immunoglobulin construct comprising a complete heavy chain, a complete light chain and a second heavy chain Fc region paired with the Fc region of the complete heavy chain. A linker can, but need not, be employed to join the heavy chain Fc region and the second heavy chain Fc region. In various embodiments, the hemibody is a monovalent antigen binding protein comprising (i) an intact light chain, and (ii) a heavy chain fused to an Fc region (e.g., an IgG2 Fc region). Methods for preparing hemibodies are described in, e.g., U.S. patent application 2012/0195879, herein incorporated by reference in its entirety herein for purposes of teaching the preparation of such hemibodies.

### Methods of Treatment

Due to their interaction with the glucagon receptor, the present antigen binding and antagonizing proteins are useful for lowering blood glucose levels by regulating gluconeogenesis and glycogenlysis and also for the treatment of a wide range of conditions and disorders in which blocking the interaction of glucagon with its receptor is beneficial, while also reducing and or eliminating one or more of the unwanted side effects associated with the current treatments.

An antagonistic antigen binding protein, in particular a human antibody according to the present disclosure, need not effect a complete cure, or eradicate every symptom or manifestation of a disease, to constitute a viable therapeutic agent. As is recognized in the pertinent field, drugs employed as therapeutic agents may reduce or ameliorate the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method comprising administering to a subject an isolated antagonistic antigen binding protein such as a human antibody in an amount and for a time sufficient to induce a sustained improvement over baseline of an indicator that reflects the severity of the particular disorder.

Thus, in one aspect, the present disclosure comprises a method for treating a rare genetic disorder in a subject, comprising administering to the subject a therapeutically effective amount of an isolated antagonistic antigen binding protein that specifically binds to the human glucagon receptor. In various embodiments, the rare genetic disorder is a disorder associated with, or characterized, in part, by abnormal resistance to insulin and/or hyperglycemia.

In various embodiments, the rare genetic disorder is selected from the group consisting of Maturity Onset Diabetes of the Young Syndrome (MODY), Wolfram Syndrome, Neonatal Diabetes, Maternally Inherited Diabetes and Deafness Syndrome, Prader-Willi Syndrome, Rabson-Mendenhall Syndrome, Insulin-resistant Acanthosis Nigricans (IRAN), Type A, Leprechaunism, Lipodystrophies, Fanconi Bickel Syndrome (FBS), Friedreich Ataxia, Klinefelter's Syndrome, Laurence-Moon Syndrome. In various embodiments, the rare genetic disorder is a ciliopathy selected from the group consisting of Alström syndrome (ALMS), Bardet-Biedl syndrome (BBS), nephronophthisis (NPHP), Senior-Löken syndrome (SNLS), Meckel syndrome (MKS), Joubert syndrome (JBTS), Oral-facial-digital Type I (OFD 1), Jeune asphyxiating thoracic dystrophy (JATD), Ellis van Creveld (EVC), Leber congenital amaurosis (LCA), autosomal recessive polycystic kidney disease (ARPKD) and autosomal dominant polycystic kidney disease (ADPKD).

In one embodiment, the present disclosure comprises treating a subject having Maturity Onset Diabetes of the Young Syndrome (MODY), comprising administering to the subject a therapeutically effective amount of an isolated antagonistic antigen binding protein that specifically binds to the human glucagon receptor.

In one embodiment, the present disclosure comprises treating a subject having Wolfram Syndrome, comprising administering to the subject a therapeutically effective amount of an isolated antagonistic antigen binding protein that specifically binds to the human glucagon receptor.

In one embodiment, the present disclosure comprises treating a subject having Neonatal Diabetes, comprising administering to the subject a therapeutically effective amount of an isolated antagonistic antigen binding protein that specifically binds to the human glucagon receptor.

In one embodiment, the present disclosure comprises treating a subject having Maternally Inherited Diabetes and Deafness Syndrome, comprising administering to the subject a therapeutically effective amount of an isolated antagonistic antigen binding protein that specifically binds to the human glucagon receptor.

In one embodiment, the present disclosure comprises treating a subject having Prader-Willi Syndrome, comprising administering to the subject a therapeutically effective amount of an isolated antagonistic antigen binding protein that specifically binds to the human glucagon receptor.

In one embodiment, the present disclosure comprises treating a subject having Rabson-Mendenhall Syndrome, comprising administering to the subject a therapeutically effective amount of an isolated antagonistic antigen binding protein that specifically binds to the human glucagon receptor.

In one embodiment, the present disclosure comprises treating a subject having Insulin-resistant Acanthosis Nigricans (IRAN), Type A, comprising administering to the subject a therapeutically effective amount of an isolated antagonistic antigen binding protein that specifically binds to the human glucagon receptor.

In one embodiment, the present disclosure comprises treating a subject having Leprechaunism, comprising administering to the subject a therapeutically effective amount of an isolated antagonistic antigen binding protein that specifically binds to the human glucagon receptor.

In one embodiment, the present disclosure comprises treating a subject having Lipodystrophies, comprising administering to the subject a therapeutically effective amount of an isolated antagonistic antigen binding protein that specifically binds to the human glucagon receptor.

In one embodiment, the present disclosure comprises treating a subject having Fanconi Bickel Syndrome (FBS), comprising administering to the subject a therapeutically effective amount of an isolated antagonistic antigen binding protein that specifically binds to the human glucagon receptor.

In one embodiment, the present disclosure comprises treating a subject having Friedreich Ataxia, comprising administering to the subject a therapeutically effective amount of an isolated antagonistic antigen binding protein that specifically binds to the human glucagon receptor.

In one embodiment, the present disclosure comprises treating a subject having Klinefelter's Syndrome, comprising administering to the subject a therapeutically effective amount of an isolated antagonistic antigen binding protein that specifically binds to the human glucagon receptor.

In one embodiment, the present disclosure comprises treating a subject having Laurence-Moon Syndrome, comprising administering to the subject a therapeutically effective amount of an isolated antagonistic antigen binding protein that specifically binds to the human glucagon receptor.

In one embodiment, the present disclosure comprises treating a subject having Alström syndrome (ALMS), comprising administering to the subject a therapeutically effective amount of an isolated antagonistic antigen binding protein that specifically binds to the human glucagon receptor.

In one embodiment, the present disclosure comprises treating a subject having Bardet-Biedl syndrome (BBS), comprising administering to the subject a therapeutically effective amount of an isolated antagonistic antigen binding protein that specifically binds to the human glucagon receptor.

In one embodiment, the present disclosure comprises treating a subject having nephronophthisis (NPHP), comprising administering to the subject a therapeutically effective amount of an isolated antagonistic antigen binding protein that specifically binds to the human glucagon receptor.

In one embodiment, the present disclosure comprises treating a subject having Senior-Löken syndrome (SNLS), comprising administering to the subject a therapeutically effective amount of an isolated antagonistic antigen binding protein that specifically binds to the human glucagon receptor.

In one embodiment, the present disclosure comprises treating a subject having Meckel syndrome (MKS), comprising administering to the subject a therapeutically effective amount of an isolated antagonistic antigen binding protein that specifically binds to the human glucagon receptor.

In one embodiment, the present disclosure comprises treating a subject having Joubert syndrome (JBTS), comprising administering to the subject a therapeutically effective amount of an isolated antagonistic antigen binding protein that specifically binds to the human glucagon receptor.

In one embodiment, the present disclosure comprises treating a subject having Oral-facial-digital Type I (OFD 1), comprising administering to the subject a therapeutically effective amount of an isolated antagonistic antigen binding protein that specifically binds to the human glucagon receptor.

In one embodiment, the present disclosure comprises treating a subject having Jeune asphyxiating thoracic dystrophy (JATD), comprising administering to the subject a therapeutically effective amount of an isolated antagonistic antigen binding protein that specifically binds to the human glucagon receptor.

In one embodiment, the present disclosure comprises treating a subject having Ellis van Creveld (EVC), comprising administering to the subject a therapeutically effective amount of an isolated antagonistic antigen binding protein that specifically binds to the human glucagon receptor.

In one embodiment, the present disclosure comprises treating a subject having Leber congenital amaurosis (LCA), comprising administering to the subject a therapeutically effective amount of an isolated antagonistic antigen binding protein that specifically binds to the human glucagon receptor.

In one embodiment, the present disclosure comprises treating a subject having autosomal recessive polycystic kidney disease (ARPKD), comprising administering to the subject a therapeutically effective amount of an isolated antagonistic antigen binding protein that specifically binds to the human glucagon receptor.

In one embodiment, the present disclosure comprises treating a subject having autosomal dominant polycystic kidney disease (ADPKD), comprising administering to the subject a therapeutically effective amount of an isolated antagonistic antigen binding protein that specifically binds to the human glucagon receptor.

In another aspect, the present disclosure comprises a method for managing and/or ameliorating one or more symptoms associated with a rare genetic disorder in a subject, comprising administering to the subject a therapeutically effective amount of an isolated antagonistic antigen binding protein that specifically binds to the human glucagon receptor. In various embodiments, the rare genetic disorder is a disorder characterized, in part, by abnormal resistance to insulin and/or hyperglycemia.

In various embodiments, the one or more symptoms is selected from the group consisting of obesity, acute or chronic hyperglycemia, hyperinsulinemia, hyperlipidemia, steatohepatitis, excess gluconeogenesis, excess glycogenolysis, ketosis, diabetic ketoacidosis, hypertriglyceridemia, elevated plasma free fatty acids, hypertension, diabetic nephropathy, renal insufficiency, renal failure, hyperphagia, muscle wasting, diabetic neuropathy, diabetic retinopathy, or diabetic coma, excess HbA1c levels, polyuria (frequent urination), polydipsia (increased thirst), xerostomia (dry mouth), polyphagia (increased hunger), fatigue, and kidney dialysis. In various embodiments, the symptom is acute or chronic hyperglycemia. In various embodiments, the symptom is hyperglucagonemia. In various embodiments, the symptom is hyperinsulinemia.

In one embodiment, the present disclosure comprises ameliorating acute or chronic hyperglycemia in a subject having Maturity Onset Diabetes of the Young Syndrome (MODY), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Wolfram Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Neonatal Diabetes, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Maternally Inherited Diabetes and Deafness Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Prader-Willi Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Rabson-Mendenhall Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Insulin-resistant Acanthosis Nigricans (IRAN), Type A, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Leprechaunism, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Lipodystrophies, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Fanconi Bickel Syndrome (FBS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Friedreich Ataxia, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Klinefelter's Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Laurence-Moon Syndrome, comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Alström syndrome (ALMS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Bardet-Biedl syndrome (BBS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having nephronophthisis (NPHP), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Senior-Löken syndrome (SNLS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Meckel syndrome (MKS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Joubert syndrome (JBTS), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Oral-facial-digital Type I (OFD 1), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Jeune asphyxiating thoracic dystrophy (JATD), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Ellis van Creveld (EVC), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having Leber congenital amaurosis (LCA), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having autosomal recessive polycystic kidney disease (ARPKD), comprising administering to the subject a therapeutically effective amount of an isolated antagonistic antigen binding protein that specifically binds to the human glucagon receptor.

In one embodiment, the present disclosure comprises ameliorating acute or chronic hyperglycemia in a subject having autosomal dominant polycystic kidney disease (ADPKD), comprising administering to the subject a therapeutically effective amount of an isolated antagonistic antigen binding protein that specifically binds to the human glucagon receptor.

### Combination Therapy

As used herein, the terms “co-administration”, “co-administered” and “in combination with”, referring to the isolated antagonistic antigen binding protein of the present disclosure and one or more other therapeutic agent(s), is intended to mean, and does refer to and include the following: simultaneous administration of such combination of isolated antagonistic antigen binding protein of the disclosure and therapeutic agent(s) to a subject in need of treatment, when such components are formulated together into a single dosage form which releases said components at substantially the same time to said subject; substantially simultaneous administration of such combination of isolated antagonistic antigen binding protein of the disclosure and therapeutic agent(s) to a subject in need of treatment, when such components are formulated apart from each other into separate dosage forms which are taken at substantially the same time by said subject, whereupon said components are released at substantially the same time to said subject; sequential administration of such combination of isolated antagonistic antigen binding protein of the disclosure and therapeutic agent(s) to a subject in need of treatment, when such components are formulated apart from each other into separate dosage forms which are taken at consecutive times by said subject with a significant time interval between each administration, whereupon said components are released at substantially different times to said subject; and sequential administration of such combination of isolated antagonistic antigen binding protein of the disclosure and therapeutic agent(s) to a subject in need of treatment, when such components are formulated together into a single dosage form which releases said components in a controlled manner whereupon they are concurrently, consecutively, and/or overlappingly released at the same and/or different times to said subject, where each part may be administered by either the same or a different route. These various combination therapies may provide a “synergistic effect”, i.e., the effect achieved when the active ingredients used together is greater than the sum of the effects that results from using the compounds separately.

Thus, in another aspect, the present disclosure comprises a method for treating a rare genetic disorder in a subject, comprising administering to the subject (a) a therapeutically effective amount of an isolated antagonistic antigen binding protein that specifically binds to the human glucagon receptor, and (b) a second therapeutic agent. In various embodiments, the combination of isolated antagonistic antigen binding protein and second therapeutic agent has a synergistic effect. In various embodiments, the second therapeutic agent is an anti-obesity agent. In various embodiments, the second therapeutic agent is a glucose lowering agent selected from the group consisting of anti-diabetic agents, anti-hyperglycemic agents, lipid lowering agents, and anti-hypertensive agents.

In another aspect, the present disclosure comprises a method for managing and/or ameliorating one or more symptoms associated with a rare genetic disorder in a subject, comprising administering to the subject (a) a therapeutically effective amount of an isolated antagonistic antigen binding protein that specifically binds to the human glucagon receptor, and (b) a second therapeutic agent. In various embodiments, the combination of isolated antagonistic antigen binding protein and second therapeutic agent has a synergistic effect. In various embodiments, the second therapeutic agent is an anti-obesity agent. In various embodiments, the second therapeutic agent is a glucose lowering agent selected from the group consisting of anti-diabetic agents, anti-hyperglycemic agents, lipid lowering agents, and anti-hypertensive agents.

Suitable anti-obesity agents (some of which may also act as anti-diabetic agents as well) include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists such as velneperit), PYY₃₋₃₆ (including analogs thereof), BRS3 modulator, mixed antagonists of opiod receptor subtypes, thyromimetic agents, dehydroepiandrosterone or an analog thereof, glucocorticoid agonists or antagonists, orexin antagonists, glucagon-like peptide-1 agonists, ciliary neurotrophic factors (such as AXOKINE™ available from Regeneron Pharmaceuticals, Inc., Tarrytown, N. Y. and Procter & Gamble Company, Cincinnati, Ohio), human agouti-related protein (AGRP) inhibitors, histamine 3 antagonists or inverse agonists, neuromedin U agonists, MTP/ApoB inhibitors (e.g., gut-selective MTP inhibitors, such as dirlotapide, JTT130, Usistapide, SLx4090), opioid antagonist, mu opioid receptor modulators, including but not limited to GSK1521498, MetAp2 inhibitors, including but not limited to ZGN-433, agents with mixed modulatory activity at 2 or more of glucagon, GIP and GLP1 receptors, such as MAR-701 or ZP2929, norepinephrine transporter inhibitors, cannabinoid-1-receptor antagonist/inverse agonists, ghrelin agonists/antagonists, oxyntomodulin and analogs, monoamine uptake inhibitors, such as but not limited to tesofensine, an orexin antagonist, combination agents (such as bupropion plus zonisamide, pramlintide plus metreleptin, bupropion plus naltrexone, phentermine plus topiramate), and the like.

In various embodiments, the anti-obesity agent is selected from gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indo1-3-ylmethyl)-5-oxo-1-phenyl-4,5-dihydro-2,3,6,10b-tetraaza-benzo[e]azulen-6-yl]-N-isopropyl-acetamide (described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (SYMLIN™), tesofensine (NS2330), leptin, bromocriptine, orlistat, AOD-9604 (CAS No. 221231-10-3) and sibutramine.

Suitable glucose-lowering agents include anti-diabetic agents, anti-hyperglycemic agents, lipid lowering agents, and anti-hypertensive agents. In various embodiments, the glucose-lowering agent is selected from biguanides, sulfonylureas, meglitinides, thiazolidinediones (TZDs), α-glucosidase inhibitors, DPP-4 inhibitors, bile acid sequestrants, dopamine-2 agonists, SGLT2 inhibitors, GLP-1 R agonists and GLP-1 agonists (e.g., exenatide (tradename Byetta®, Amylin/Astrazeneca); liraglutide (tradename Victoza®, Novo Nordisk A/S), lixisenatide (tradename Lyxumia®, Sanofi); albiglutide (tradename Tanzeum®, GlaxoSmithKline); dulaglutide (tradename Trulicity®, Eli Lilly)), amylin mimetics, and insulins. Such agents have been extensively described in the art (see, e.g., lnzucchi et al., Diabetologia, 58:429-442, 2015). In various embodiments, the DPP-4 inhibitor is selected from Alogliptin/Nesina, Gemigliptin, Teneligliptin, Melogliptin, Trelagliptin, Trajenta/Linagliptin/BI-1356/Ondero/Trajenta/Tradjenta/Trayenta/Tradzenta- , Saxagliptin/Onglyza, Sitagliptin/Januvia/Xelevia/Tesave/Janumet/Velmetia, Galvus/Vildagliptin, Anagliptin, DA-1229, Omarigliptin/MK-3102, KM-223, Evogliptin, ARI-2243, PBL-1427, and Pinoxacin. In various embodiments, the SGLT2 inhibitor is selected from, e.g., lnvokana/Canaglifozin, Forxiga/Dapagliflozin, Farxiga, Remoglifozin, Sergliflozin, Empagliflozin, Ipragliflozin, Tofogliflozin, Luseogliflozin, LX-4211, Ertuglifozin/PF-04971729, RO-4998452, EGT-0001442, KGA-3235/DSP-3235, LIK066, and SBM-TFC-039. In various embodiments, the biguanide is selected from, e.g., Metformin, Buformin, and Phenformin. In various embodiments, the thiazolidinedione is selected from, e.g., Pioglitazone, Rivoglitazone, Rosiglitazone, and Troglitazone. In various embodiments the sulfonylurea is selected from, e.g., Tolbutamide, Glibenclamide, Glimepiride/Amaryl, and Glipizide. In various embodiments, the meglitinide is selected from, e.g., Nateglinide, Repaglinide, and Mitiglinide. In various embodiments, the amylin and amylin mimetic is selected from, e.g., Pramlintide, and Symlin™.

An illustrative, but not limiting, list of suitable glucose metabolism-related proteins to be used as the glucose-lowering agent in the combination therapies of the present disclosure is provided in Table 3:

TABLE 3

| Glucose metabolism-related | |
|---|---|
| proteins | RefSeq (NCBI/Uniprot) |
| Glucagon proprotein | NP_002045.1 |
| Glucagon peptide | NP_002045.1 (aa 53-81) |
| Glucagon-like peptide 1 | NP_002045.1 (aa 98-127) |
| Glucagon-like peptide 2 | NP_002045.1 (aa 146-178) |
| Glicentin | P01275 (aa 21-89) |
| Glicentin-related polypeptide | P01275 (aa 21-50) |
| Gastric inhibitory polypeptide | NP_004114.1 |
| preprotein | |
| Gastric inhibitory polypeptide | NP_004114.1 (aa 52-93) |
| Dipeptidyl peptidase 4 | P27487 |
| Glucose transporter member 4 | NP_001033.1 |
| Preproglucagon | AAA52567.1 |
| Insulin receptor substrate 1 | NP_005535.1 |
| Human Insulin Preprotein | NP_000198.1 |
| Apolipoprotein A-II | P02652 |
| Solute carrier family 2, facilitated | P11166 |
| glucose transporter member 1 | |
| Glycogen synthase 1 | P13807 |
| Glycogen synthase 2 | P54840 |
| Tyrosin-protein phosphatase non- | P18031 |
| receptor type 1 | |
| RAC-alpha serine threonine-protein | P31749 |
| kinase | |
| Peroxisome proliferator-activated | P37231 |
| receptor gamma | |
| Hexokinase 3 | P52790 |
| Phosphatidylinositol-3,4,5- | P60484 |
| triphosphate 3-phosphatase and dual- | |
| specificity protein | |
| Pyruvate dehydrogenase kinase 1 | Q15118 |
| Calcium-binding and coiled-coil | Q9P1Z2 |
| domain-containing protein 1 | |
| Max-like protein X | Q9UH92 |
| Fructose-bisphosphate aldolase A | P04075 |
| Glucagon-like peptide 1 receptor | P43220 |
| Glucagon-like peptide 2 receptor | O95838 |
| Gastric inhibitory polypeptide receptor | P48546 |
| Insulin-like growth factor 1 receptor | P08069.1 |
| Insulin-like growth factor 2 receptor | P11717.3 |
| Insulin Receptor | P06213 |
| GLP-1 agonist-Exenatide | DB01276 |
| GLP-1 agonist-Liraglutide | DB06655 |
| Exendin-4 | GenBank: AAA70888.1 |
| Exendin-3 | GenBank: AAA70887.1 |
| GIPR agonist (Des-Ala²-GIP₁₋₃₀) | NP_004114.1 (aa 52-81) |
| GIPR agonist-Truncated GIP₁₋₃₀ | NP_004114.1 (aa 52-81) |
| GLP-1R agonist (aa 1-37 of GIP) | NP_004114.1 (aa 52-88) |
| GLP-1R agonist (aa 7-36 of GIP) | NP_004114.1 (aa 58-87) |

In various embodiments, the glucose lowering agent is selected from a GLP-1 R agonist and GLP-1 agonist (e.g., exenatide (tradename Byetta®, Amylin/Astrazeneca).

Insulin and insulin analogs/derivatives have been extensively described in the art (see e.g., US20150216981; U.S. Pat. No. 9,265,723; U.S. Pat. No. 8,633,156; U.S. Pat. No. 8,410,048; U.S. Pat. No. 8,048,854; U.S. Pat. No. 7,713,930; U.S. Pat. No. 7,696,162; U.S. Pat. No. 7,659,363; U.S. Pat. No. 7,291,132; U.S. Pat. No. 7,193,035; and references cited therein, which are all incorporated by reference herein). In various embodiments, the insulin or insulin anaolog/derivative is selected from the group consisting of Glargine/Lantus®, Glulisin/Apidra®, Glarine/Toujeo®, Insuman®, Detemir/Levemir®, Lispro/Humalog®/Liprolog®, Degludec/DegludecPlus, Aspart, basal insulin and analogues (e.g. LY-2605541, LY2963016, NN1436), PEGylated insulin Lispro, Humulin®, Linjeta, SuliXen®, NN1045, Insulin plus SymlinTM, PE0139, fast-acting and short-acting insulins (e.g. Linjeta, PH20, NN1218, HinsBet), (APC-002) hydrogel, oral, inhalable, transdermal and sublingual insulins (e.g. Exubera®, Nasulin®, Afrezza®, Tregopil®, TPM 02, Capsulin, Oral-lyn®, Cobalamin®, oral insulin, ORMD-0801, NN1953, NN1954, NN1956, VIAtab, and Oshadi oral insulin). In various embodiments, the insulin co-administration comprises administering a dose of insulin or insulin derivative that may be between about 70%-90%, between about 50%-70%, between about 30%-50%, between about 15%-30%, between about 10-15%, between about 5-10%, and between zero and 5%, including 4%, 3.5%, 3%, 2.5%, 2%, 1.5%, 1%, 0.5%, 0.4%, 0.3%, 0.2%, or 0.1% of the normal daily dosage of insulin.

In various embodiments, the combination therapy comprises administering the isolated antagonistic antigen binding protein composition and the second agent composition simultaneously, either in the same pharmaceutical composition or in separate pharmaceutical compositions. In various embodiments, isolated antagonistic antigen binding protein composition and the second agent composition are administered sequentially, i.e., the isolated antagonistic antigen binding protein composition is administered either prior to or after the administration of the second agent composition.

In various embodiments, the administrations of the isolated antagonistic antigen binding protein composition and the second agent composition are concurrent, i.e., the administration period of the isolated antagonistic antigen binding protein composition and the second agent composition overlap with each other.

In various embodiments, the administrations of the isolated antagonistic antigen binding protein composition and the second agent composition are non-concurrent. For example, in various embodiments, the administration of the isolated antagonistic antigen binding protein composition is terminated before the second agent composition is administered. In various embodiments, the administration second agent composition is terminated before the isolated antagonistic antigen binding protein composition is administered.

### Dosing Regimens

A therapeutically effective dose can be estimated initially from cell culture assays by determining an IC₅₀. A dose can then be formulated in animal models to achieve a circulating plasma concentration range that includes the IC₅₀ as determined in cell culture. Such information can be used to more accurately determine useful doses in humans. Levels in plasma may be measured by, e.g., HPLC or immunoassays using the anti-idiotypic antibodies specific to the therapeutic drug. The exact composition, route of administration and dosage can be chosen by the individual physician in view of the subject's condition.

Dosage regimens can be adjusted to provide the optimum desired response (e.g., a therapeutic or prophylactic response). For example, a single bolus can be administered, several divided doses (multiple or repeat or maintenance) can be administered over time and the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the present disclosure will be dictated primarily by the unique characteristics of the antibody and the particular therapeutic or prophylactic effect to be achieved.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present disclosure.

It is to be noted that dosage values may vary with the type and severity of the condition to be ameliorated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Further, the dosage regimen with the compositions of this disclosure may be based on a variety of factors, including the type of disease, the age, weight, sex, medical condition of the subject, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present disclosure encompasses intra-subject dose-escalation as determined by the skilled artisan. Determining appropriate dosages and regimens are well-known in the relevant art and would be understood to be encompassed by the skilled artisan once provided the teachings disclosed herein.

For administration to human subjects, the total monthly dose of the isolated antagonistic antigen binding protein of the disclosure can be in the range of 0.5-1200 mg per subject, 0.5-1100 mg per subject, 0.5-1000 mg per subject, 0.5-900 mg per subject, 0.5-800 mg per subject, 0.5-700 mg per subject, 0.5-600 mg per subject, 0.5-500 mg per subject, 0.5-400 mg per subject, 0.5-300 mg per subject, 0.5-200 mg per subject, 0.5-100 mg per subject, 0.5-50 mg per subject, 1-1200 mg per subject, 1-1100 mg per subject, 1-1000 mg per subject, 1-900 mg per subject, 1-800 mg per subject, 1-700 mg per subject, 1-600 mg per subject, 1-500 mg per subject, 1-400 mg per subject, 1-300 mg per subject, 1-200 mg per subject, 1-100 mg per subject, or 1-50 mg per subject depending, of course, on the mode of administration. For example, an intravenous monthly dose can require about 1-1000 mg/subject. In certain embodiments, the isolated antagonistic antigen binding protein of the disclosure can be administered at about 1-200 mg per subject, 1-150 mg per subject or 1-100 mg per subject. The total monthly dose can be administered in single or divided doses and can, at the physician's discretion, fall outside of the typical ranges given herein.

In various embodiments, the isolated antagonistic antigen binding protein of the disclosure is administered to the subject at a dosage (e.g., at a weekly dosage) included in any of the following ranges: about 5 to about 15 mg, about 15 to about 30 mg, about 30 to about 45 mg, about 45 to about 60 mg, about 60 to about 75 mg, about 75 to about 90 mg, about 90 to about 105 mg, about 105 to about 120 mg, about 120 to about 135 mg, about 135 to about 150 mg, about 150 to about 165 mg, about 165 to about 180 mg, about 180 to about 195 mg, and about 195 to about 210 mg. In various embodiments, the isolated antagonistic antigen binding protein of the disclosure is administered to the subject at a weekly dosage selected from the group consisting of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 200 mg, 205 mg, and 210 mg.

In various embodiments, the total dose administered will achieve a plasma antibody concentration in the range of, e.g., about 1 to 1000 μg/ml, about 1 to 750 μg/ml, about 1 to 500 μg/ml, about 1 to 250 μg/ml, about 10 to 1000 μg/ml, about 10 to 750 μg/ml, about 10 to 500 μg/ml, about 10 to 250 μg/ml, about 20 to 1000 μg/ml, about 20 to 750 μg/ml, about 20 to 500 μg/ml, about 20 to 250 μg/ml, about 30 to 1000 μg/ml, about 30 to 750 μg/ml, about 30 to 500 μg/ml, about 30 to 250 μg/ml.

An exemplary, non-limiting monthly dosing range for a therapeutically or prophylactically effective amount of an isolated antagonistic antigen binding protein of the disclosure can be 0.001 to 10 mg/kg, 0.001 to 9 mg/kg, 0.001 to 8 mg/kg, 0.001 to 7 mg/kg, 0.001 to 6 mg/kg, 0.001 to 5 mg/kg, 0.001 to 4 mg/kg, 0.001 to 3 mg/kg, 0.001 to 2 mg/kg, 0.001 to 1 mg/kg, 0.010 to 10 mg/kg, 0.010 to 9 mg/kg, 0.010 to 8 mg/kg, 0.010 to 7 mg/kg, 0.010 to 6 mg/kg, 0.010 to 5 mg/kg, 0.010 to 4 mg/kg, 0.010 to 3 mg/kg, 0.010 to 2 mg/kg, 0.010 to 1 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, 0.1 to 1 mg/kg, 1 to 10 mg/kg, 1 to 9 mg/kg, 1 to 8 mg/kg, 1 to 7 mg/kg, 1 to 6 mg/kg, 1 to 5 mg/kg, 1 to 4 mg/kg, 1 to 3 mg/kg, 1 to 2 mg/kg, or 1 to 1 mg/kg body weight. It is to be noted that dosage values may vary with the type and severity of the condition to be ameliorated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In various embodiments, the isolated antagonistic antigen binding protein of the disclosure is administered to the subject at a dosage (e.g., at a weekly dosage) included in any of the following ranges: about 0.1 to about 0.2 mg/kg, about 0.2 to about 0.4 mg/kg, about 0.4 to about 0.6 mg/kg, about 0.6 to about 0.8 mg/kg, about 0.8 to about 1.0 mg/kg, about 1.0 to about 1.2 mg/kg, about 1.2 to about 1.4 mg/kg, about 1.4 to about 1.6 mg/kg, about 1.6 to about 1.8 mg/kg, about 1.8 to about 2.0 mg/kg, about 2.0 to about 2.2 mg/kg, about 2.2 to about 2.4 mg/kg, about 2.4 to about 2.6 mg/kg, about 2.6 to about 2.8 mg/kg, and about 2.8 to about 3.0 mg/kg. In various embodiments, the isolated antagonistic antigen binding protein of the disclosure is administered to the subject at a weekly dosage selected from the group consisting of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, and 3.0 mg/kg.

In various embodiments, either as monotherapy, or in combination with a second therapeutic agent, the weekly or bi-weekly dose for a therapeutically effective amount of an isolated antagonistic antigen binding protein of the disclosure will be 0.01 mg/kg body weight. In various embodiments, the weekly or bi-weekly dose for a therapeutically effective amount of an isolated antagonistic antigen binding protein of the disclosure will be 0.025 mg/kg body weight. In various embodiments, the weekly or bi-weekly dose for a therapeutically effective amount of an isolated antagonistic antigen binding protein of the disclosure will be 0.05 mg/kg body weight. In various embodiments, the weekly or bi-weekly dose for a therapeutically effective amount of an isolated antagonistic antigen binding protein of the disclosure will be 0.075 mg/kg body weight. In various embodiments, the weekly or bi-weekly dose for a therapeutically effective amount of an isolated antagonistic antigen binding protein of the disclosure will be 0.1 mg/kg body weight. In various embodiments, the weekly or bi-weekly dose for a therapeutically effective amount of an isolated antagonistic antigen binding protein of the disclosure will be 0.25 mg/kg body weight. In various embodiments, the weekly or bi-weekly dose for a therapeutically effective amount of an isolated antagonistic antigen binding protein of the disclosure will be 0.5 mg/kg body weight. In various embodiments, the weekly or bi-weekly dose for a therapeutically effective amount of an isolated antagonistic antigen binding protein of the disclosure will be 0.75 mg/kg body weight. In various embodiments, the weekly or bi-weekly dose for a therapeutically effective amount of an isolated antagonistic antigen binding protein of the disclosure will be 1 mg/kg body weight. In various embodiments, the weekly or bi-weekly dose for a therapeutically effective amount of an isolated antagonistic antigen binding protein of the disclosure will be 1.5 mg/kg body weight. In various embodiments, the weekly or bi-weekly dose for a therapeutically effective amount of an isolated antagonistic antigen binding protein of the disclosure will be 2 mg/kg body weight. In various embodiments, the weekly or bi-weekly dose for a therapeutically effective amount of an isolated antagonistic antigen binding protein of the disclosure will be 2.5 mg/kg body weight. In various embodiments, the weekly or bi-weekly dose for a therapeutically effective amount of an isolated antagonistic antigen binding protein of the disclosure will be 3 mg/kg body weight. In various embodiments, the weekly or bi-weekly dose for a therapeutically effective amount of an isolated antagonistic antigen binding protein of the disclosure will be 3.5 mg/kg body weight. In various embodiments, the weekly or bi-weekly dose for a therapeutically effective amount of an isolated antagonistic antigen binding protein of the disclosure will be 4 mg/kg body weight. In various embodiments, the weekly or bi-weekly dose for a therapeutically effective amount of an isolated antagonistic antigen binding protein of the disclosure will be 4.5 mg/kg body weight. In various embodiments, the weekly or bi-weekly dose for a therapeutically effective amount of an isolated antagonistic antigen binding protein of the disclosure will be 5 mg/kg body weight. In various embodiments, the weekly or bi-weekly dose for a therapeutically effective amount of an isolated antagonistic antigen binding protein of the disclosure will be 5.5 mg/kg body weight. In various embodiments, the weekly or bi-weekly dose for a therapeutically effective amount of an isolated antagonistic antigen binding protein of the disclosure will be 6 mg/kg body weight. In various embodiments, the weekly or bi-weekly dose for a therapeutically effective amount of an isolated antagonistic antigen binding protein of the disclosure will be 6.5 mg/kg body weight. In various embodiments, the weekly or bi-weekly dose for a therapeutically effective amount of an isolated antagonistic antigen binding protein of the disclosure will be 7 mg/kg body weight. In various embodiments, the weekly or bi-weekly dose for a therapeutically effective amount of an isolated antagonistic antigen binding protein of the disclosure will be 7.5 mg/kg body weight. In various embodiments, the weekly or bi-weekly dose for a therapeutically effective amount of an isolated antagonistic antigen binding protein of the disclosure will be 8 mg/kg body weight. In various embodiments, the weekly or bi-weekly dose for a therapeutically effective amount of an isolated antagonistic antigen binding protein of the disclosure will be 8.5 mg/kg body weight. In various embodiments, the weekly or bi-weekly dose for a therapeutically effective amount of an isolated antagonistic antigen binding protein of the disclosure will be 9 mg/kg body weight. In various embodiments, the weekly or bi-weekly dose for a therapeutically effective amount of an isolated antagonistic antigen binding protein of the disclosure will be 9.5 mg/kg body weight. In various embodiments, the weekly or bi-weekly dose for a therapeutically effective amount of an isolated antagonistic antigen binding protein of the disclosure will be 10 mg/kg body weight.

An isolated antagonistic antigen binding protein that specifically binds the human glucagon receptor, in particular, the fully human antibodies of the disclosure, may be administered, e.g., once or more than once, at regular intervals over a period of time. In various embodiments, a fully human antibody is administered over a period of at least once a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the fully human antibody is administered until the subject manifests a medically relevant degree of improvement over baseline for the chosen indicator or indicators.

One example of therapeutic regimens provided herein comprise subcutaneous injection of an isolated antagonistic antigen binding protein once a week, or once every two weeks, at an appropriate dosage, to treat a condition in which blood glucose levels play a role. Weekly or monthly administration of isolated antagonistic antigen binding protein would be continued until a desired result is achieved, e.g., the subject's symptoms subside. Treatment may resume as needed, or, alternatively, maintenance doses may be administered.

A subject's levels of blood glucose may be monitored before, during and/or after treatment with an isolated antagonistic antigen binding protein such as a human antibody, to detect changes, if any, in their levels. For some disorders, the incidence of elevated blood glucose may vary according to such factors as the stage of the disease. Known techniques may be employed for measuring glucose levels. Glucagon levels may also be measured in the subject's blood using known techniques, for example, ELISA.

Toxicity and therapeutic index of the pharmaceutical compositions of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effective dose is the therapeutic index and it can be expressed as the ratio LD₅₀/ED₅₀. Compositions that exhibit large therapeutic indices are generally preferred.

In various embodiments, single or multiple administrations of the pharmaceutical compositions are administered depending on the dosage and frequency as required and tolerated by the subject. In any event, the composition should provide a sufficient quantity of at least one of the isolated antagonistic antigen binding protein disclosed herein to effectively treat the subject. The dosage can be administered once but may be applied periodically until either a therapeutic result is achieved or until side effects warrant discontinuation of therapy.

The dosing frequency of the administration of the isolated antagonistic antigen binding protein pharmaceutical composition depends on the nature of the therapy and the particular disease being treated. The subject can be treated at regular intervals, such as weekly or monthly, until a desired therapeutic result is achieved. Exemplary dosing frequencies include, but are not limited to: once weekly without break; once weekly, every other week; once every 2 weeks; once every 3 weeks; weakly without break for 2 weeks, then monthly; weakly without break for 3 weeks, then monthly; monthly; once every other month; once every three months; once every four months; once every five months; or once every six months, or yearly.

### Pharmaceutical Compositions

In another aspect, the present disclosure provides a pharmaceutical composition comprising an isolated antagonistic antigen binding protein as described herein, and a second therapeutic agent, with one or more pharmaceutically acceptable carrier(s). The pharmaceutical compositions and methods of uses described herein also encompass embodiments of combinations (co-administration) with other active agents, as detailed below. The isolated antagonistic antigen binding proteins provided herein can be formulated by a variety of methods apparent to those of skill in the art of pharmaceutical formulation. Such methods may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

Generally, the antagonistic antigen binding proteins of the present disclosure are suitable to be administered as a formulation in association with one or more pharmaceutically acceptable carrier(s). The term ‘carrier’ is used herein to describe any ingredient other than the compound(s) of the disclosure. The choice of carrier(s) will to a large extent depend on factors such as the particular mode of administration, the effect of the carrier on solubility and stability, and the nature of the dosage form. As used herein, “pharmaceutically acceptable carrier” includes any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, the composition will include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Pharmaceutical compositions of the present disclosure and methods for their preparation will be readily apparent to those skilled in the art. Such compositions and methods for their preparation may be found, for example, in Remington's Pharmaceutical Sciences, 19th Edition (Mack Publishing Company, 1995). The pharmaceutical compositions are generally formulated as sterile, substantially isotonic and in full compliance with all GMP regulations of the U.S. Food and Drug Administration.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute thereof. In one embodiment of the present disclosure, compositions may be prepared for storage by mixing the selected composition having the desired degree of purity with optional formulation agents (Remington's Pharmaceutical Sciences, supra) in the form of a lyophilized cake or an aqueous solution. Further, the therapeutic composition may be formulated as a lyophilizate using appropriate excipients such as sucrose. The optimal pharmaceutical composition will be determined by one of ordinary skill in the art depending upon, for example, the intended route of administration, delivery format, and desired dosage.

The pharmaceutical compositions of the present disclosure are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous injection, intraperitoneal injection, intramuscular injection, intrasternal injection, intravenous injection, intraarterial injection, intrathecal injection, intraventricular injection, intraurethral injection, intracranial injection, intrasynovial injection or infusions; or kidney dialytic infusion techniques.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded. Numerous reusable pen and autoinjector delivery devices have been described in the literature and are commercially available.

Formulations of a pharmaceutical composition suitable for parenteral administration typically generally comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition. Parenteral formulations also include aqueous solutions which may contain carriers such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating the isolated antagonistic antigen binding protein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation such as vacuum drying and freeze-drying yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin. In various embodiments, the injectable compositions will be administered using commercially available disposable injectable devices.

The isolated antagonistic antigen binding protein of the present disclosure can be administered intranasally or by inhalation, typically in the form of a dry powder (either alone, as a mixture, or as a mixed component particle, for example, mixed with a suitable pharmaceutically acceptable carrier) from a dry powder inhaler, as an aerosol spray from a pressurized container, pump, spray, atomiser (preferably an atomiser using electrohydrodynamics to produce a fine mist), or nebulizer, with or without the use of a suitable propellant, or as nasal drops.

The pressurized container, pump, spray, atomizer, or nebulizer generally contains a solution or suspension of an isolated antagonistic antigen binding protein of the disclosure comprising, for example, a suitable agent for dispersing, solubilizing, or extending release of the active, a propellant(s) as solvent.

Prior to use in a dry powder or suspension formulation, the drug product is generally micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules, blisters and cartridges for use in an inhaler or insufflator may be formulated to contain a powder mix of the isolated antagonistic antigen binding protein of the disclosure, a suitable powder base and a performance modifier.

Suitable flavours, such as menthol and levomenthol, or sweeteners, such as saccharin or saccharin sodium, may be added to those formulations of the disclosure intended for inhaled/intranasal administration.

Formulations for inhaled/intranasal administration may be formulated to be immediate and/or modified release. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the disclosure are typically arranged to administer a metered dose or “puff” of an antibody of the disclosure. The overall daily dose will typically be administered in a single dose or, more usually, as divided doses throughout the day.

The isolated antagonistic antigen binding protein of the present disclosure may also be formulated for an oral administration. Oral administration may involve swallowing, so that the compound enters the gastrointestinal tract, and/or buccal, lingual, or sublingual administration by which the compound enters the blood stream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Pharmaceutical compositions intended for oral use may be prepared according to any method known to the art for the manufacture of pharmaceutical compositions and such compositions may contain one or more agents selected from the group consisting of sweetening agents in order to provide a pharmaceutically elegant and palatable preparation. For example, to prepare orally deliverable tablets, the isolated antagonistic antigen binding protein is mixed with at least one pharmaceutical carrier, and the solid formulation is compressed to form a tablet according to known methods, for delivery to the gastrointestinal tract. The tablet composition is typically formulated with additives, e.g. a saccharide or cellulose carrier, a binder such as starch paste or methyl cellulose, a filler, a disintegrator, or other additives typically usually used in the manufacture of medical preparations. To prepare orally deliverable capsules, DHEA is mixed with at least one pharmaceutical carrier, and the solid formulation is placed in a capsular container suitable for delivery to the gastrointestinal tract. Compositions comprising isolated antagonistic antigen binding protein may be prepared as described generally in Remington's Pharmaceutical Sciences, 18th Ed. 1990 (Mack Publishing Co. Easton Pa. 18042) at Chapter 89, which is herein incorporated by reference.

In various embodiments, the pharmaceutical compositions are formulated as orally deliverable tablets containing isolated antagonistic antigen binding protein in admixture with non-toxic pharmaceutically acceptable carriers which are suitable for manufacture of tablets. These carriers may be inert diluents, such as calcium carbonate, sodium carbonate, lactose, calcium phosphate or sodium phosphate; granulating and disintegrating agents, for example, maize starch, gelatin or acacia, and lubricating agents, for example, magnesium stearate, stearic acid, or talc. The tablets may be uncoated or they may be coated with known techniques to delay disintegration and absorption in the gastrointestinal track and thereby provide a sustained action over a longer period of time. For example, a time delay material such as glyceryl monostearate or glyceryl distearate alone or with a wax may be employed.

In various embodiments, the pharmaceutical compositions are formulated as hard gelatin capsules wherein the isolated antagonistic antigen binding protein is mixed with an inert solid diluent, for example, calcium carbonate, calcium phosphate, or kaolin or as soft gelatin capsules wherein the isolated antagonistic antigen binding protein is mixed with an aqueous or an oil medium, for example, arachis oil, peanut oil, liquid paraffin or olive oil.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Any method for administering peptides, proteins or antibodies accepted in the art may suitably be employed for administering the isolated antagonistic antigen binding protein of the disclosure.

The invention having been described, the following examples are offered by way of illustration, and not limitation.

### EXAMPLE 1

In this example, the therapeutic potential of an antagonistic monoclonal antibody against the glucagon receptor was evaluated in an ALMS mouse model. Specifically, the in vivo activity of an anti-GCGR antibody which comprises the heavy chain sequence set forth in SEQ ID NO: 8 and the light chain sequence set forth in SEQ ID NO: 9 (“REMD2.59C) was evaluated using the “Alms1 gene-trapped mice” model of ALMS. Alms1 gene-trapped mice (Alms1^{-/-}) are generated by inserting a mutation into the Alms1 gene in exon 13 leading to the premature truncation of the Alms1 protein. Alms1^{-/-} mice also exhibit similar metabolic disturbances to those of human patients with ALMS, such as obesity, hyperglycemia, hyper insulinemia, steatohepatitis, infertility and sensorineuronal hearing loss (Collin et al., Hum. Mol. Genet., 14: 2323-2333, 2005).

The study involved 5 treatment groups: Group I: 4 week old Alms1^{-/-} mice (n=10) are dosed once a week up until week 20 via intraperitoneal injection with placebo (10 mM sodium acetate, 5% sorbitol, and 0.004% polysorbate 20); Group II: 4 week old Alms1^{-/-} mice (n=10) are dosed once a week up until week 20 via intraperitoneal injection with 7.5 mg/kg REMD2.59C antibody; Group III: 4 week old control littermates (“wildtype”)(n=10) are dosed once a week up until week 20 via intraperitoneal injection with placebo (10 mM sodium acetate, 5% sorbitol, and 0.004% polysorbate 20); Group IV: 4 week old control littermates (“wildtype”) (n=10) are dosed once a week up until week 20 via intraperitoneal injection with 7.5 mg/kg REMD2.59C antibody; Group V: 10 week old Alms1^{-/-} mice (n=8) are dosed once a week up until week 20 via intraperitoneal injection with 7.5 mg/kg REMD2.59C antibody; and Group VI: 10 week old control littermates (“wildtype”)(n=8) are dosed once a week up until week 20 via intraperitoneal injection with 7.5 mg/kg REMD2.59C antibody. Groups I-IV are referred to as “prevention” groups. Groups V-VI are referred to as “treatment” groups.

EDTA whole blood samples were collected at week 8 and week 20 for all groups. Heparinized plasma were collected at week 8, week 12, week 16 and week 20 for Groups I-IV and at week 10, week 12, week 16 and week 20 for Group V. The injection day was Monday of each week and the collection day was Thursday for each of the respective collection weeks. Various parameters are determined at week 8, week 12, week 16 and week 20 of the study: i) fasting blood glucose determinations (mice are fasted for 6 hours prior to the test); ii) serum alanine aminotransferase (ALT) determinations; iii) aspartate aminotransferase (AST) determinations; and iv) serum lipids (e.g., total cholesterol (CHO), LDL, HDL and triglycerides (TG)) determinations. At week 8 and week 20, serum hemoglobin-A1c (HbA1c) determinations and blood plasma glucagon determinations are made. At week 20, serum 3-HB (Hydroxybutyrate) determinations are made and oral glucose tolerance test (OGTT) is performed (1g glucose/kg body weight) with one touch glucose levels determined at t=0, 15, 30, 60, and 120 minutes. The following day, final REMD and/or injections are administered, and 2 days later final bleed and necropsy performed. Liver, kidney and heart tissue specimens are collected at the end of the study for all treatment groups.

Table 4 depicts the fasting glucose levels (mg/dl) for various mice determined at week 20.

TABLE 4

| | | Clinical | Fasting Glucose |
|---|---|---|---|
| Mouse | Genotype | Treatment | (mg/dl) |
| Group I | Alms1^{-/-} | placebo | >600 |
| 6697 | | | |
| Group I | Alms1^{-/-} | placebo | 347 |
| 6691 | | | |
| Group I | Alms1^{-/-} | placebo | 489 |
| 6692 | | | |
| Group II | Alms1^{-/-} | REMD Ab | 154 |
| 6664 | | | |
| Group II | Alms1^{-/-} | REMD Ab | 180 |
| 6666 | | | |
| Group II | Alms1^{-/-} | REMD Ab | 187 |
| 6705 | | | |
| Group III | wildtype | placebo | 97 |
| 6699 | | | |
| Group IV | wildtype | REMD Ab | 94 |
| 6663 | | | |
| Group IV | wildtype | REMD Ab | 130 |
| 6665 | | | |
| Group V | Alms1^{-/-} | REMD Ab | 118 |
| 6655 | | | |
| Group V | Alms1^{-/-} | REMD Ab | 92 |
| 6649 | | | |
| Group VI | wildtype | REMD Ab | 87 |
| 6658 | | | |

As depicted in Table 4, treatment using REMD 2.59 Ab has a marked effect on lowering fasting glucose levels in both the prevention group (Group II) and treatment group (Group V), with levels in the treatment group approaching the levels observed in the wildtype mice.

Table 5 depicts the results of the OGTT analysis performed at week 20. Following oral administration of 1 g/kg glucose, the blood glucose levels were measured at different time points (0, 15, 30, 60, 120 min).

TABLE 5

| | | | Glucose | Glucose | Glucose | Glucose | Glucose |
|---|---|---|---|---|---|---|---|
| | | Clinical | (mg/dl) | (mg/dl) | (mg/dl) | (mg/dl) | (mg/dl) |
| Mouse | Genotype | Treatment | 0 mins | 15 mins | 30 mins | 60 mins | 120 mins |
| Group I | Alms1^{-/-} | placebo | >600 | >600 | >600 | >600 | >600 |
| 6697 | | | | | | | |
| Group I | Alms1^{-/-} | placebo | 283 | 436 | 426 | 407 | 236 |
| 6691 | | | | | | | |
| Group I | Alms1^{-/-} | placebo | 362 | 445 | 492 | 526 | 313 |
| 6692 | | | | | | | |
| Group II | Alms1^{-/-} | REMD Ab | 165 | 277 | 325 | 264 | 215 |
| 6664 | | | | | | | |
| Group II | Alms1^{-/-} | REMD Ab | 133 | 278 | 383 | 331 | 207 |
| 6666 | | | | | | | |
| Group III | wildtype | placebo | 106 | 132 | 166 | 124 | 102 |
| 6699 | | | | | | | |
| Group IV | wildtype | REMD Ab | 99 | 146 | 201 | 138 | 98 |
| 6663 | | | | | | | |
| Group IV | wildtype | REMD Ab | 119 | 162 | 169 | 143 | 81 |
| 6665 | | | | | | | |
| Group V | Alms1^{-/-} | REMD Ab | 132 | 157 | 205 | 192 | 138 |
| 6655 | | | | | | | |
| Group V | Alms1^{-/-} | REMD Ab | 103 | 145 | 158 | 110 | 80 |
| 6649 | | | | | | | |
| Group VI | wildtype | REMD Ab | 107 | 132 | 143 | 129 | 80 |
| 6658 | | | | | | | |

The Table 5 data demonstrates that the REMD 2.59 Ab treated Alms1^{-/-} mice (prevention Group II and treatment Group V) have superior OGTT profiles as compared to the placebo treated Alms1^{-/-} mice, with levels in the treatment group approaching the levels observed in the wildtype mice.

Collectively, the data in Tables 4 and 5 demonstrate that administration of REMD 2.59 Ab is capable of lowering blood glucose levels in both the prevention and treatment groups, and that REMD 2.59 Ab treatment provides for an improved OGTT profile. Accordingly, it has been demonstrated that the treatment of ALMS mice using an anti-GCGR antibody provides the beneficial therapeutic effect of ameliorating or preventing hyperglycemia is the ALMS mice.

### EXAMPLE 2

In this example, the in vivo activity of an anti-GCGR antibody which comprises the heavy chain sequence set forth in SEQ ID NO: 8 and the light chain sequence set forth in SEQ ID NO: 9 (“REMD2.59C”) is evaluated using the “fat aussie” mouse model of ALMS (Arsuv et al., Mol. Endocrinol., 20: 1610-1622, 2006).

The fat aussie mouse carries a spontaneous mutation (Alms1^{foz}) in Alms1 that was detected as the sudden appearance of obese offspring in a nonobese diabetic mouse colony. The Alms1^{foz} mutation consists of a small (11 bp) deletion in exon 8 that causes a frameshift and a premature stop codon. This mutation is similar in exon location and type to ALMS1 mutations mapped in human patients with ALMS, suggesting that exon 8 of ALMS1 forms a ‘mutational hotspot’. Owing to the premature stop codon, the alms1 protein in fat aussie mice is truncated, missing approximately two-thirds of its C-terminal length. Homozygous fat aussie mice (Alms1^{foz/foz}) demonstrate most of the clinical features of ALMS seen in human patients, including obesity, hyperglycemia, hyperinsulinemia, hyperlipidemia, steatohepatitis, infertility and sensorineuronal hearing loss (Arsuv et al., Mol. Endocrinol., 20: 1610-1622, 2006).

The mice are dosed weekly or bi-weekly via subcutaneous injection with either vehicle (10 mM sodium acetate, 5% sorbitol, and 0.004% polysorbate 20), 2.5 mg/kg REMD2.59C antibody (“Low Dose”), 5 mg/kg REMD2.59C antibody (“Middle Dose”), or 7.5 mg/kg REMD2.59C antibody (“High Dose”) for 24 weeks. The dose administered will not exceed 10mg/kg per month. Various parameters are measured throughout the study, including, e.g., i) body weight (once a week); ii) fasting blood glucose determination (mice are fasted for 6 hours prior to the test and fasting blood glucose levels are measured via tail veins weekly using Accu-Chek Aviva System®; iii) serum hemoglobin-A1c (HbA1c) determination; iv) serum GLP-1 determination; v) serum insulin and leptin levels via radioimmunoassay (Linco, St. Charles, Mo.); vi) serum alanine aminotransferase (ALT) determination; vii) serum adioponectin determination; viii) serum lipids (e.g., total cholesterol (CHO), LDL, HDL and triglycerides (TG)) determination; and ix) gamma-glutamyl transpeptidase (CGT) determination . For items iii)-ix), blood samples are collected pre-dose and at the end of the study into tubes without any anticoagulant, immediately centrifuged and the serum transferred into separate sample tubes for evaluation. At the end of the study, liver, kidney and heart tissue specimens are collected for all treatment groups.

It is expected that treatment of the fat aussie mice using an anti-GCGR antibody will provide beneficial therapeutics effects such as, e.g., preventing hyperglycemia, preventing hyperglucagonemia, reducing insulin resistance; reducing insulin insufficiency; reducing or preventing hyperinsulinemia, reducing or preventing fat deposits in the liver; reducing or preventing inflammation in the liver; reducing or preventing the accumulation of lipid, e.g., hepatic triacylglycerol, hepatic diacylglycerol, and ceramides; and preventing injury in the liver.

### EXAMPLE 3

This Example describes a clinical study to evaluate the safety, pharmacokinetics and pharmacodynamic effects of weekly treatment using a fully human anti-GCGR antibody in subjects diagnosed with ALMS. The treatment may last a period up to 6 or 12 months, long enough to observe and quantitate treatment efficacy and safety.

Treatment groups include a placebo group and treatment groups to be treated with various dosages of a fully human anti-GCGR antibody which comprises the heavy chain sequence set forth in SEQ ID NO: 51 and the light chain sequence set forth in SEQ ID NO: 52 (“REMD-477”). Examples of non-placebo treatment groups may include, e.g., subjects who receive injections of either 0.01 mg/kg, 0.025 mg/kg, 0.05 mg/kg, 0.075 mg/kg, 0.1 mg/kg, 0.25 mg/kg, 0.5 mg/kg, 0.75 mg/kg, 1.0 mg/kg, 1.5 mg/kg, 2.0 mg/kg, 2.5 mg/kg, 5 mg/kg, 7.5 mg/kg, or 10 mg/kg REMD-477 per week, or bi-weekly.

Primary outcome measures will include, e.g., effects on obesity, change in fasting plasma glucose levels, average of daily morning glucose after an overnight fast, at pre-treatment baseline, and at weekly intervals until the end of study; change in plasma insulin levels at pre-treatment baseline, and at weekly intervals until the end of study; change in hemoglobin A1c levels (indicator of chronic glucose control) at pre-treatment baseline, and at 4-week or 8-week intervals until the end of study; change in glucose profiles at oral glucose tolerance tests (OGTT), measured at 0, 30, 60, 90 and 120 min after an oral glucose load, at pre-treatment baseline, and at 8-week intervals until the end of study.

Secondary outcome measures will include, e.g., change in percentage of liver fat content by MRI at: baseline, at 4-week or 8-week intervals, until the end of study; change in the proportion of REMD-477 treated patients relative to placebo achieving improvement of liver fibrosis by at least one stage, at the end of study, in comparison to the baseline assessment; and change in liver enzyme and metabolic markers, including Aspartate Transaminase (AST), Alanine Transaminase (ALT), Bilirubin and Alkaline phosphatase (ALP), at: baseline, at 4-week intervals, until the end of study; composite long term outcome measured by the number of patients with the onset of any adjudicated events, including cirrhosis, all-cause mortality, and liver-related clinical outcomes, at the Baseline and the end of study; and changes in scores of the Quality of Life.

### Additional Materials and Methods

Blood Glucose: The mice were fasted 6 hours prior to blood glucose test from 9 am to 3 pm, and fast blood glucose levels were measured via tail veins on weekly basis by using Accu-Chek Aviva System.

Oral Glucose Tolerance Test: To test the repeat dosing effect of REMD Ab2.59, OGTT was performed for all animals at the end of the study. The baseline (time 0) glucose level was measured after 16 hours fast and prior to glucose challenge. Following oral administration of 1 g/kg glucose, the blood glucose levels were measured at different time points (0, 15, 30, 60, 120 min) by using Accu-Chek Performa System.

Blood Biochemistry: EDTA whole blood samples are collected at week 8 and week 20 for all groups. Heparinized plasma are collected at week 8, week 12, week 16 and week 20 for Groups I-IV and at week 10, week 12, week 16 and week 20 for Group V. The whole blood samples were used for analysis of the serum levels of HbA1c at week 8 and week 20. The plasma-heparin samples are used for analysis of ALT, AST, cholesterol, free fatty acids, glucose and triglycerides levels at week 8, 12, 16 and 20.

Liver Weight: At the end of the study, livers are rapidly excised, rinsed in ice-cold saline, and weighed. Aliquots of liver are snap frozen in liquid nitrogen and kept at −80° C. until being analyzed. A portion of each liver is fixed in 10% formalin for histology.

Histology and immunohistochemistry: At the end of the study, heart, kidney and livers are rapidly excised, rinsed in ice-cold saline, and weighed. For the histological studies, the heart and kidney are excised and fixed using Bouin's fixative. The liver is excised and liver necropsy (on medial lobe) fixed in Tellys. The rest of the liver is kept at −80° C. for lipid profile analysis.

All of the articles and methods disclosed and claimed herein can be made and executed without undue experimentation in light of the present disclosure. While the articles and methods of this disclosure have been described in terms of preferred embodiments, it will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

### Sequence Listings

The instant application contains a Sequence Listing in the form of a “paper copy” (PDF File) and a file containing the referenced sequences (SEQ D NOs: 1-52) in computer readable form (ST25 format text file) which is submitted herein. The amino acid sequences listed in the accompanying sequence listing are shown using standard three letter code for amino acids, as defined in 37 C.F.R. 1.822.

SEQ ID NO: 1 is the amino acid sequence of a human glucagon receptor (GCGR) molecule (Accession Number AA104855).

SEQ ID NO: 2 is the amino acid sequence encoding the heavy chain variable region of a fully human anti-GCGR antibody. SEQ ID NO: 3 is the amino acid sequence encoding the light chain variable region of a fully human anti-GCGR antibody.

SEQ ID NO: 4 is the amino acid sequence encoding the heavy chain variable region of a fully human anti-GCGR antibody. SEQ ID NO: 5 is the amino acid sequence encoding the light chain variable region of a fully human anti-GCGR antibody.

SEQ ID NO: 6 is the amino acid sequence encoding the heavy chain variable region of a fully human anti-GCGR antibody. SEQ ID NO: 7 is the amino acid sequence encoding the light chain variable region of a fully human anti-GCGR antibody.

SEQ ID NO: 8 is the amino acid sequence encoding the heavy chain of a chimeric anti-GCGR antibody. SEQ ID NO: 9 is the amino acid sequence encoding the light chain of a chimeric anti-GCGR antibody.

SEQ ID NOS: 10-28 are amino acid sequences encoding the heavy chain variable regions of various fully human anti-GCGR antibodies.

SEQ ID NOS: 29-47 are amino acid sequences encoding the light chain variable regions of various fully human anti-GCGR antibodies.

SEQ ID NO: 48 is the amino sequence encoding a kappa light chain constant region.

SEQ ID NO: 49 is the amino sequence encoding a lambda light chain constant region.

SEQ ID NO: 50 is the amino sequence encoding a IgG2 heavy chain constant region.

SEQ ID NO: 51 is the amino acid sequence encoding the heavy chain of a human anti-GCGR antibody. SEQ ID NO: 52 is the amino acid sequence encoding the light chain of a human anti-GCGR antibody.

SEQUENCE LISTINGS

SEQ ID NO: 1-Amino acid sequence of a human glucagon receptor (GCGR) molecule
MPPCQPQRPLLLLLLLLACQPQVPSAQVMDFLFEKWKLYGDQCHHNLSLLPPPTELVCNRTFD
KYSCWPDTPANTTANISCPWYLPWHHKVQHRFVFKRCGPDGQWVRGPRGQPWRDASQCQ
MDGEEIEVQKEVAKMYSSFQVMYTVGYSLSLGALLLALAILGGLSKLHCTRNAIHANLFASFVLK
ASSVLVIDGLLRTRYSQKIGDDLSVSTWLSDGAVAGCRVAAVFMQYGIVANYCWLLVEGLYLH
NLLGLATLPERSFFSLYLGIGWGAPMLFVVPWAVVKCLFENVQCWTSNDNMGFWWILRFPVFL
AILINFFIFVRIVQLLVAKLRARQMHHTDYKFRLAKSTLTLIPLLGVHEVVFAFVTDEHAQGTLRSA
KLFFDLFLSSFQGLLVAVLYCFLNKEVQSELRRRWHRWRLGKVLWEERNTSNHRASSSPGHG
PPSKELQFGRGGGSQDSSAETPLAGGLPRLAESPF
SEQ ID NO: 2-Amino acid sequence of a HCVR of a human antibody that binds GCGR
QVQLVESGGGVVQPGRSLRLSCAASGFTFSSYGMHWVRQAPGKGLEWVAVMWYD
GSNKDYVDSVKGRFTISRDNSKNTLYLQMNRLRAEDTAVYYCAREKDHYDILTGYN
YYYGLDVWGQGTTVTVSS
SEQ ID NO: 3-Amino acid sequence of a LCVR of a human antibody that binds GCGR
DIQMTQSPSSLSASVGDRVTITCRASQGIRNDLGWYQQKPGKAPKRLIYAASSLQSGV
PSRFSGSGSGTEFTLTISSVQPEDFVTYYCLQHNSNPLTFGGGTKVEIK
SEQ ID NO: 4-Amino acid sequence of a HCVR of a human antibody that binds GCGR
QVQLVESGGGVVQPGRSLRLSCAASGFTFSSYGMHWVRQAPGKGLEWV
AVMWYDGSNKDYVDSVKGRFTISRDNSKNTLYLQMNSLRAEDTAVYYCAREKDHYDI
LTGYNYYYGLDVWGQGTTVTVSS
SEQ ID NO: 5-Amino acid sequence of a LCVR of a human antibody that binds GCGR
DIQMTQSPSSLSASVGDRVTITCRASQGIRNDLGWYQQKPGKAPKRLIYAASSLQSGV
PSRFSGSGSGTEFTLTISSLQPEDFVTYYCLQHNSNPLTFGGGTKVEIK
SEQ ID NO: 6-Amino acid sequence of a HCVR of a human antibody that binds GCGR
QVQLVESGGGVVQPGRSLRLSCAASGFTFSSYGMHWVRQAPGKGLEWVAVMWYD
GSNKDYVDSVKGRFTISRDNSKNTLYLQMNRLRAEDTAVYYCAREKDHYDILTGYNY
YYGLDVWGQGTTVTVSS
SEQ ID NO: 7-Amino acid sequence of a LCVR of a human antibody that binds GCGR
DIQMTQSPSSLSASVGDRVTITCRASQGIRNDLGWYQQKPGKAPKRLIYAASSLESGV
PSRFSGSGSGTEFTLTISSVQPEDFVTYYCLQHNSNPLTFGGGTKVEIK
SEQ ID NO: 8-Amino acid sequence of a heavy chain of a chimeric antibody that binds GCGR
MEFGLSWVFLVALLRGVQCQVQLVESGGGVVQPGRSLRLSCAASGFTFSSYGMHWVRQAPG
KGLEWVAVMWYDGSNKDYVDSVKGRFTISRDNSKNTLYLQMNRLRAEDTAVYYCAREKDHY
DILTGYNYYYGLDVWGQGTTVTVSSAKTTPPSVYPLAPGSAAQTNSMVTLGCLVKGYFPEPVT
VTWNSGSLSSGVHTFPAVLQSDLYTLSSSVTVPSSTWPSETVTCNVAHPASSTKVDKKIVPRD
CGCKPCICTVPEVSSVFIFPPKPKDVLTITLTPKVTCVVVDISKDDPEVQFSWFVDDVEVHTAQT
QPREEQFNSTFRSVSELPIMHQDWLNGKEFKCRVNSAAFPAPIEKTISKTKGRPKAPQVYTIPP
PKEQMAKDKVSLTCMITDFFPEDITVEWQWNGQPAENYKNTQPIMDTDGSYFVYSKLNVQKS
NWEAGNTFTCSVLHEGLHNHHTEKSLSHSPGK
SEQ ID NO: 9-Amino acid sequence of a light chain of a chimeric antibody that binds GCGR
MDMRVPAQLLGLLLLWFPGARCDIQMTQSPSSLSASVGDRVTITCRASQGIRNDLGWYQQKP
GKAPKRLIYAASSLESGVPSRFSGSGSGTEFTLTISSVQPEDFVTYYCLQHNSNPLTFGGGTKV
EIKRADAAPTVSIFPPSSEQLTSGGASVVCFLNNFYPKDINVKWKIDGSERQNGVLNSWTDQDS
KDSTYSMSSTLTLTKDEYERHNSYTCEATHKTSTSPIVKSFNRNEC
SEQ ID NO: 10-Amino acid sequence of a HCVR of a human antibody that binds GCGR
QVQLVESGGGVVQPGRSLRLSCAASGFTFSNYGMHWVRQAPGKGLEWVAVILSDGRNKYYA
DSVKGRFTISRDNSKNTLYLQMNSLRAEDTAVYYCARDDYEILTGYGYYGMDVWGQGTTVTV
SS
SEQ ID NO: 11-Amino acid sequence of a HCVR of a human antibody that binds GCGR
QVQLVESGGGVVQPGRSLRLSCAASGFTFSSYGMHWVRQAPGKGLEWVAVILNDGRNKYYA
DSVKGRFTISRDNSKNTLYLQMNSLRAEDTAVYYCARDDYEILTGYGYYGMDVWGQGTTVTV
SS
SEQ ID NO: 12-Amino acid sequence of a HCVR of a human antibody that binds GCGR
QVQLQQSGPGLVKPSQTLSLTCAISGDSVSSNGAAWNWIRQSPSRGLEWLGRTYYRSKWYY
DYAGSVKSRININPDTSKNQFSLQVNSVTPEDTAVYYCTRDRSSGWNEGYYYYGMDVWGQG
TTVTVSS
SEQ ID NO: 13-Amino acid sequence of a HCVR of a human antibody that binds GCGR
QVQLVESGGGVVQPGRSLRLSCAASGFTFSSYDIHWVRQAPGKGLEWVAVLSSDGNNKYCA
DSVKGRFTISRDNSKNTLYLQMNSLRTEDTAVYYCAREEVYYDILTGYYDYYGMDVWGQGTTV
TVSS
SEQ ID NO: 14-Amino acid sequence of a HCVR of a human antibody that binds GCGR
QVQLQESGPGLVKPSETLSLTCTVSGGSISTYFWTWIRQFPGKGLEWIGYIFYSGSTNYNPSLK
SRVTISVDTSKNQFSLKLSSVTAADTAVYYCAREGYYDILTGEDYSYGMDVWGQGTTVTVSS
SEQ ID NO: 15-Amino acid sequence of a HCVR of a human antibody that binds GCGR
QVQLQQSGPGLVKPSQILSLICAISGDRVSSNGAAWNWIRQSPSRGLEWLGRTYYRSKWYYD
YAGSVKSRININPDTSKNQFSLQVNSVTPEDTAVYYCARDRSSGWNEGYYYYGMDVWGQGT
TVTVSS
SEQ ID NO: 16-Amino acid sequence of a HCVR of a human antibody that binds GCGR
QVQLQESGPGLVKPSETLSLTCTVSGGSISTYFWTWIRQFPGEGLEWIGYIFYSGNTNYNPSLT
SRVTISVDTSKNQFSLKLSSVTAADTAVYYCAREGYYDILTGEDYSYGIDVWGQGTTVTVSS
SEQ ID NO: 17-Amino acid sequence of a HCVR of a human antibody that binds GCGR
QVQLVESGGGVVQPGRSLRLSCAASGFIFSSYGMHWVRQAPGKGLEWVAVISNDGSNKYYA
DFVKGRFTISRDNSKNTLYLQMNSLRAEDTAVYYCAREDYDILTGNGVYGMDVWGQGTTVTV
SS
SEQ ID NO: 18-Amino acid sequence of a HCVR of a human antibody that binds GCGR
EVQLVESGGGLVQPGGSLRLSCAASGFTFSSYTMNWVRQAPGKGLEWVSYISGSSSLIYYAD
SVKGRFTISRDNAKNSLYLHMNSLRDEDTAVYYCARARYNWNDYYGMDVWGQGTTVTVSS
SEQ ID NO: 19-Amino acid sequence of a HCVR of a human antibody that binds GCGR
QVQLVESGGGVVQPGRSLRLSCAASGFAFSSYGIHWVRQAPGKGLEWVAGIWYDGSNKYYA
DSVKGRFTVSRDNSKNTLYLQMNSLRAEDTAVYYCARLFDAFDIWGQGTMVTVSS
SEQ ID NO: 20-Amino acid sequence of a HCVR of a human antibody that binds GCGR
EVQLVESGGGLVQPGGSLRLSCAASGFIFSSYTMNWVRQAPGKGLEWVSYISSSSSLIYYADS
VKGRFTISRDNAKNSLYLQMNSLRDEDTAVYYCARSDYYGSGSYYKGNYYGMDVWGQGTTV
TVSS
SEQ ID NO: 21-Amino acid sequence of a HCVR of a human antibody that binds GCGR
QVQLVESGGGVVQPGRSLRLSCAASGFTFSSYGMHWVRQAPGKGLEWVTIIWSDGINKYYAD
SVKGRFTISRDNSKNTLNLQMNSLRAEDTAVYYCARERGLYDILTGYYDYYGIDVWGQGTTVT
VSS
SEQ ID NO: 22-Amino acid sequence of a HCVR of a human antibody that binds GCGR
QVQLVESGGGVVQPGRSLRLSCAASGFTFSSYGMHWVRQAPGKGLEWVTIIWSDGINKYYAD
SVKGRFTISRDNSKNTLNLQMNSLRAEDTAVYYCARERGLYDILTGYYDYYGIDVWGQGTTVT
VSS
SEQ ID NO: 23-Amino acid sequence of a HCVR of a human antibody that binds GCGR
EVQLVESGGGLVKPGGSLRLSCAASGITFRSYSMNWVRQAPGKGLEWVSAISSSSSYIYYADS
VKGRFTISRDNAKNSVYLQMNSLRAEDTAVYYCARGRYGMDVWGQGTTVTVSS
SEQ ID NO: 24-Amino acid sequence of a HCVR of a human antibody that binds GCGR
QVQLVESGGGVVQPGRSLRLSCAASGSTFRSYDMHWVRQAPGKGLEWVAVISYDGSNKYYG
DSVKGRLTISRDNSKNTLYLQMNSLRAEDTAVYYCARDQYDILTGYSSDAFDIWGQGTMVTV
SS
SEQ ID NO: 25-Amino acid sequence of a HCVR of a human antibody that binds GCGR
QVQLVESGGGVVQPGRSLRLSCAASGFTFSRYGMHWVRQAPGKGLEWVAVIWYDGSHKYY
EDSVKGRFTISRDNSKNTLYLQMNSLRADDTGVYYCARVGYGSGWYEYYYHYGMDVWGQGT
TVTVSS
SEQ ID NO: 26-Amino acid sequence of a HCVR of a human antibody that binds GCGR
QVQLVESGGGVVQPGRSLRLSCAASGFTFSSYGMHWVRQAPGKGLEWVAVMWYDGSNKDY
VDSVKGRFTISRDNSKNTLYLQMNRLRAEDTAVYYCAREKDHYDILTGYNYYYGLDVWGQGTT
VTVSS
SEQ ID NO: 27-Amino acid sequence of a HCVR of a human antibody that binds GCGR
QVQLVESGGGVVQPGRSLRLSCAASGFTFSSYGMHWVRQAPGKGLEWVAVMWYDGSNKDY
VDSVKGRFTISRDNSKNTLYLQMNRLRAEDTAVYYCAREKDHYDILTGYNYYYGLDVWGQGTT
VTVSS
SEQ ID NO: 28-Amino acid sequence of a HCVR of a human antibody that binds GCGR
QVQLVESGGGVVQPGRSLRLSCAASGITFSSYGMHWVRQAPGKGLEWVASIWYDGSNKYYV
DSVKGRFTIFRDNSKKTLYLQMNRLRAEDTAVYYCARLGGGFDYWGQGTLVTVSS
SEQ ID NO: 29-Amino acid sequence of a LCVR of a human antibody that binds GCGR
DIQMTQSPSSLSASVGDRVTITCRASQDISNYLAWFQKKPGKAPKSLIYVVSSLQSGVPSRFSG
SGSGTDFTLTINNLQPEDFATYYCQQYNHYPLTFGGGTRVEIKR
SEQ ID NO: 30-Amino acid sequence of a LCVR of a human antibody that binds GCGR
DIQMTQSPSSLSASVGDRVTITCRASQDISNYLAWFQQRPGKAPKSLIYVVSSLQSGVPSRFSG
SGSGTDFTLTISNLQPEDFATYFCQQYNHYPLTFGGGTKVEIKR
SEQ ID NO: 31-Amino acid sequence of a LCVR of a human antibody that binds GCGR
DIQMTQFPSSLSASIGDRVTITCQASQDISNFLNWFQQKPGKAPKLLIYDASDLETGVPSRFSGS
GAGTDFTFTISSLQPEDIATYFCQQYDDLPLTFGGGTRVDIKR
SEQ ID NO: 32-Amino acid sequence of a LCVR of a human antibody that binds GCGR
DIQMTQSPSSLSASVGDRVTITCRASQGIRNDLGWYQQKPGKAPKRLIYAASSLQSGVPSRFS
GSGSGTEFTLTISSLQPEDFATYYCLQHNSNPLTFGGGTKVEIKR
SEQ ID NO: 33-Amino acid sequence of a LCVR of a human antibody that binds GCGR
QNVLIQSPGILSLSPGERVILSCRASQSVSSSYLAWYQQKPGQAPRLLIFGVSSRATGIPDRF
SGSGSGTDFSLTISRLEPEDFAVYYCQQYGNSPFTFGPGTKVDIKR
SEQ ID NO: 34-Amino acid sequence of a LCVR of a human antibody that binds GCGR
DIQMTQFPSSLSASIGDRVTITCQASQDISNFLNWFQQKPGKAPKLLIYDASDLETGVPSRFSGS
GAGTDFTFTISSLQPEDVATYFCQQYDNLPLTFGGGTKVDIKR
SEQ ID NO: 35-Amino acid sequence of a LCVR of a human antibody that binds GCGR
ENVLTQSPGTLSLSPGERATLSCRASQSVTSSYLAWYQQKPGQAPRLLIFGVSSRATGIPDRF
SGSGSGTDFSLTISRLEPEDFAVYYCQQYGNSPFTFGPGTKVDIKR
SEQ ID NO: 36-Amino acid sequence of a LCVR of a human antibody that binds GCGR
DIQMTQSPSSLSASVGDRVTITCRASQGIDMYLAWFQQKPGKAPKSLIYAASSLQSGVPSKFS
GSGFGTDFTLTISSLQPEDFATYYCQQYNIFPFTFGPGTKVDVKR
SEQ ID NO: 37-Amino acid sequence of a LCVR of a human antibody that binds GCGR
DIQMTQSPSSLSASVGDRVTITCRASQGIRNDLGWYQQKPGKAPKRLIYAASSLESGVPSRFS
GSGSGTEFTLTISSLQPEDFATYYCLQHNSYPWTFGQGTKVEIKR
SEQ ID NO: 38-Amino acid sequence of a LCVR of a human antibody that binds GCGR
KIVMTQTPLALPVIPGEPASISCRSSQSLVDSDDGDTYLDWYLQKPGQSPQVLIHRLSYRASGV
PDRFSGSGSGTDFTLKISRVEAEDVGIYYCMHRIEFPFTFGGGTKVEIKR
SEQ ID NO: 39-Amino acid sequence of a LCVR of a human antibody that binds GCGR
DIQMTQSPSSLSASVGDRVTITCRASQGIRNDLGWYQQRPGKAPKRLIYAASSLQTGVPSRFS
GSGSGTEFTLTISSLQPEDFATYYCLQHNSYPWTFGQGTKVEIKR
SEQ ID NO: 40-Amino acid sequence of a LCVR of a human antibody that binds GCGR
GIVLTQSPLSLPVTPGEPASISCRSSQSLLHSNGYNYLDWYLQKPGQSPQLLIYLGSNRASGVP
DRFSGSGSGTDFTLKISRVEAEDVGVYYCMEALQTMCSFGQGTKLEIKR
SEQ ID NO: 41-Amino acid sequence of a LCVR of a human antibody that binds GCGR
GIVLTQSPLSLPVTPGEPASISCRSSQSLLHSNGYNYLDWYLQKPGQSPQLLIYLGSNRASGVP
DRFSGSGSGTDFTLKISRVEAEDVGVYYCMEALQTMSSFGQGTKLEIKR
SEQ ID NO: 42-Amino acid sequence of a LCVR of a human antibody that binds GCGR
DIVMTQTPLFLPVTPGEPASISCRSSQTLLDSDDGNTYLDWYLQKPGQSPQRLIYTLSYRASGV
PDRFSGSGSGTDFTLKISRVEAEDVGIYYCMQHIEFPSTFGQGTRLEIKR
SEQ ID NO: 43-Amino acid sequence of a LCVR of a human antibody that binds GCGR
SYELTQPPSVSVSPGQTASITCSGDKLGDKYASWYQQKPGQSPVLVIYQSTKRPSGIPERFSG
SNSGNTATLTISGTQAMDEADYYCQAWDSSTVVFGGGTKLTVLG
SEQ ID NO: 44-Amino acid sequence of a LCVR of a human antibody that binds GCGR
NIVMTQTPLSLSVTPGQPASISCKSSQSLLHSDGKNYLFWYLQKPGQSPQLLIYEVSYRFSGVP
DRFSGSGSGTDFSLKISRVEAEDVGVYYCMQNIQPPLIFGQGTRLEIKR
SEQ ID NO: 45-Amino acid sequence of a LCVR of a human antibody that binds GCGR
DIQMTQSPSSLSASVGDRVTITCRASQGIRNDLGWYQQKPGKAPKRLIYAASSLQSGVPSRFS
GSGSGTEFTLTISSVQPEDFVTYYCLQHNSNPLTFGGGTKVEIKR
SEQ ID NO: 46-Amino acid sequence of a LCVR of a human antibody that binds GCGR
DIQMTQSPSSLSASVGDRVTITCRASQGIRNDLGWYQQKPGKAPKRLIYAASSLESGVPSRFS
GSGSGTEFTLTISSVQPEDFVTYYCLQHNSNPLTFGGGTKVEIKR
SEQ ID NO: 47-Amino acid sequence of a LCVR of a human antibody that binds GCGR
DIVLTQTPLSLPVTPGEPASISCRSSQSLLDRDDGDTYLDWYLQKPGQSPQLLIYTLSYRASGV
PDRFSGSGSGTDFSLKISRVEAEDVGVYYCMQRIEFPFTFGPGTKVDIKR
SEQ ID NO: 48-Amino acid sequence of the constant light chain kappa region
RTVAAPSVFIFPPSDEQLKSGTASVVCLLNNFYPREAKVQWKVDNALQSGNSQESVTEQDSKD
STYSLSSTLTLSKADYEKHKVYACEVTHQGLSSPVTKSFNRGEC
SEQ ID NO: 49-Amino acid sequence of the constant light chain lambda region
GQPKAAPSVTLFPPSSEELQANKATLVCLISDFYPGAVTVAWKADSSPVKAGVETTTPSKQSN
NKYAASSYLSLTPEQWKSHRSYSCQVTHEGSTVEKTVAPTECS
SEQ ID NO: 50-Amino sequence of the IgG2 heavy chain constant region
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSHEDPEVQFNWYVDGVEVHNAKTKPREEQFNSTFRVVSVLTVVH
QDWLNGKEYKCKVSNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVFSCSVMHEALHNH
YTQKSLSLSPGK
SEQ ID NO: 51-Amino acid sequence of a HC of a human antibody that binds GCGR
QVQLVESGGGVVQPGRSLRLSCAASGFTFSSYGMHWVRQAPGKGLEWVAVMWYD
GSNKDYVDSVKGRFTISRDNSKNTLYLQMNRLRAEDTAVYYCAREKDHYDILTGYN
YYYGLDVWGQGTTVTVSSASTKGPSVFPLAPCSRSTSESTAALGCLVKDYFPEPVTVSWN
SGALTSGVHTFPAVLQSSGLYSLSSVVTVPSSNFGTQTYTCNVDHKPSNTKVDKTVERKCCVE
CPPCPAPPVAGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVYTLP
PSREEMTKNQVSLTCLVKGFYPSDIAVEWESNGQPENNYKTTPPMLDSDGSFFLYSKLTVDKS
RWQQGNVFSCSVMHEALHNHYTQKSLSLSPGK
SEQ ID NO: 52-Amino acid sequence of a LC of a human antibody that binds GCGR
DIQMTQSPSSLSASVGDRVTITCRASQGIRNDLGWYQQKPGKAPKRLIYAASSLQSGV
PSRFSGSGSGTEFTLTISSVQPEDFVTYYCLQHNSNPLTFGGGTKVEIKRTVAAPSVFIF
PPSDEQLKSGTASVVCLLNNFYPREAKVQWKVDNALQSGNSQESVTEQDSKDSTYSLSSTLTL
SKADYEKHKVYACEVTHQGLSSPVTKSFNRGEC

1-20. (canceled)

21. A method for treating a rare genetic disorder in a subject, comprising administering to the subject a therapeutically effective amount of an isolated antagonistic antigen binding protein that specifically binds to the human glucagon receptor.

22. A method according to claim 21, wherein the rare genetic disorder is selected from the group consisting of Alström syndrome (ALMS), Bardet-Biedl syndrome (BBS), nephronophthisis (NPHP), Senior-Löken syndrome (SNLS), Meckel syndrome (MKS), Joubert syndrome (JBTS), Oral-facial-digital Type I (OFD 1), Jeune asphyxiating thoracic dystrophy (JATD), Ellis van Creveld (EVC), Leber congenital amaurosis (LCA), autosomal recessive polycystic kidney disease (ARPKD), autosomal dominant polycystic kidney disease (ADPKD), Maturity Onset Diabetes of the Young Syndrome (MODY), Wolfram Syndrome, Neonatal Diabetes, Maternally Inherited Diabetes and Deafness Syndrome, Prader-Willi Syndrome, Rabson-Mendenhall Syndrome, Insulin-resistant Acanthosis Nigricans (IRAN), Type A, Leprechaunism, Lipodystrophies, Fanconi Bickel Syndrome (FBS), Friedreich Ataxia, Klinefelter's Syndrome, and Laurence-Moon Syndrome.

23. A method according to claim 22, wherein the rare genetic disorder is Alström syndrome (ALMS).

24. A method according to claim 21, wherein the isolated antagonistic antigen binding protein comprises an antibody selected from the group consisting of a fully human antibody, a humanized antibody, a chimeric antibody, a monoclonal antibody, a polyclonal antibody, a recombinant antibody, an antigen-binding antibody fragment, a Fab, a Fab′, a Fab₂, a Fab′2, a IgG, a IgM, a IgA, a IgE, a scFv, a dsFv, a dAb, a nanobody, a unibody, a diabody, and a hemibody, wherein the isolated antagonistic binding protein binds to a human glucagon receptor with a dissociation constant (K_{D}) of at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

25. A method according to claim 24, wherein the isolated antagonistic antibody is a fully human antibody.

26. A method according to claim 25, wherein the fully human antagonistic antibody comprises the amino acid sequence encoding the heavy chain variable region of SEQ ID NO: 2 and the amino acid sequence encoding the light chain variable region of SEQ ID NO: 3.

27. A method according to claim 25, wherein the fully human antagonistic antibody comprises the amino acid sequence encoding the heavy chain variable region of SEQ ID NO: 4 and the amino acid sequence encoding the light chain variable region of SEQ ID NO: 5.

28. A method according to claim 25, wherein the fully human antagonistic antibody comprises the amino acid sequence encoding the heavy chain variable region of SEQ ID NO: 6 and the amino acid sequence encoding the light chain variable region of SEQ ID NO: 7.

29. A method according to claim 25, wherein the fully human antagonistic antibody comprises a heavy chain variable region having the amino acid sequence selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, and SEQ ID NO: 28 and comprises a light chain variable region having the amino acid sequence selected from the group consisting of SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, and SEQ ID NO: 47.

30. A method according to claim 29, wherein the fully human antagonistic antibody comprises the amino acid sequence encoding the heavy chain variable region of SEQ ID NO: 28 and the amino acid sequence encoding the light chain variable region of SEQ ID NO: 47.

31. A method according to claim 25, wherein the fully human antagonistic antibody comprises the amino acid sequence encoding the heavy chain of SEQ ID NO: 51 and the amino acid sequence encoding the light chain of SEQ ID NO: 52.

32. A method according to claim 24, wherein the chimeric antagonistic antibody comprises the amino acid sequence encoding the heavy chain of SEQ ID NO: 8 and the amino acid sequence encoding the light chain of SEQ ID NO: 9.

33. A method according to claim 21, wherein the therapeutically effective amount of the isolated antagonistic antigen binding protein is selected from the group consisting of 0.001 to 100 mg/kg, 0.001 to 90 mg/kg, 0.001 to 80 mg/kg, 0.001 to 70 mg/kg, 0.001 to 60 mg/kg, 0.001 to 50 mg/kg, 0.001 to 40 mg/kg, 0.001 to 30 mg/kg, 0.001 to 20 mg/kg, 0.001 to 10 mg/kg, 0.001 to 5 mg/kg, 0.001 to 4 mg/kg, 0.001 to 3 mg/kg, 0.001 to 2 mg/kg, 0.001 to 1 mg/kg, 0.010 to 50 mg/kg, 0.010 to 40 mg/kg, 0.010 to 30 mg/kg, 0.010 to 20 mg/kg, 0.010 to 10 mg/kg, 0.010 to 5 mg/kg, 0.010 to 4 mg/kg, 0.010 to 3 mg/kg, 0.010 to 2 mg/kg, 0.010 to 1 mg/kg, 0.1 to 50 mg/kg, 0.1 to 40 mg/kg, 0.1 to 30 mg/kg, 0.1 to 20 mg/kg, 0.1 to 10 mg/kg, 0.1 to 5 mg/kg, 0.1 to 4 mg/kg, 0.1 to 3 mg/kg, 0.1 to 2 mg/kg, 0.1 to 1 mg/kg, 0.5 to 50 mg/kg, 0.5 to 40 mg/kg, 0.5 to 30 mg/kg, 0.5 to 20 mg/kg, 0.5 to 10 mg/kg, 0.5 to 5 mg/kg, 0.5 to 4 mg/kg, 0.5 to 3 mg/kg, 0.5 to 2 mg/kg, 0.5 to 1 mg/kg, 1 to 50 mg/kg, 1 to 40 mg/kg, 1 to 30 mg/kg, 1 to 20 mg/kg, 1 to 10 mg/kg, 1 to 5 mg/kg, 1 to 4 mg/kg, 1 to 3 mg/kg, 1 to 2 mg/kg, and 0.1 mg/kg to 1 mg/kg body weight per week.

34. A method according to claim 33, wherein the therapeutically effective amount of the isolated antagonistic antigen binding protein is 0.01 to 10 mg/kg body weight per week.

35. A method for treating a rare genetic disorder in a subject, comprising administering to the subject (a) a therapeutically effective amount of an isolated antagonistic antigen binding protein that specifically binds to the human glucagon receptor, and (b) a second therapeutic agent.

36. A method according to claim 35, wherein the second therapeutic agent is an anti-obesity agent or a glucose lowering agent selected from the group consisting of anti-diabetic agents, anti-hyperglycemic agents, lipid lowering agents, and anti-hypertensive agents.

37. A method for ameliorating one or more symptoms associated with a rare genetic disorder in a subject, comprising administering to the subject a therapeutically effective amount of an isolated antagonistic antigen binding protein that specifically binds to the human glucagon receptor.

38. A method according to claim 37, wherein the one or more symptoms is selected from the group consisting of obesity, acute or chronic hyperglycemia, hyperinsulinemia, hyperlipidemia, steatohepatitis, excess gluconeogenesis, excess glycogenolysis, ketosis, diabetic ketoacidosis, hypertriglyceridemia, elevated plasma free fatty acids, hypertension, diabetic nephropathy, renal insufficiency, renal failure, hyperphagia, muscle wasting, diabetic neuropathy, diabetic retinopathy, or diabetic coma, excess HbA1c levels, polyuria (frequent urination), polydipsia (increased thirst), xerostomia (dry mouth), polyphagia (increased hunger), fatigue, and kidney dialysis.

39. A method according to claim 38, wherein the one or more symptom is hyperglycemia.

40. A method according to claim 21, wherein the isolated antagonistic antigen binding protein is admixed with a pharmaceutically acceptable carrier to form a pharmaceutical composition for systemic administration to the subject, wherein the systemic administration is selected from: intravenous injection, intramuscular injection, subcutaneous injection, intraperitoneal injection, transdermal injection, intraarterial injection, intrasternal injection, intrathecal injection, intraventricular injection, intraurethral injection, intracranial injection, intrasynovial injection or via infusions.


SEQUENCE LISTING (52 sequences)

SEQ ID NO: 1 - protein, 477 aa, Homo sapiens
MPPCQPQRPLLLLLLLLACQPQVPSAQVMDFLFEKWKLYGDQCHHNLSLLPPPTELVCNRTFDKYSCWPDTPANTTANISCPWYLPWHHKVQHRFVFKRCGPDGQWVRGPRGQPWRDASQCQMDGEEIEVQKEVAKMYSSFQVMYTVGYSLSLGALLLALAILGGLSKLHCTRNAIHANLFASFVLKASSVLVIDGLLRTRYSQKIGDDLSVSTWLSDGAVAGCRVAAVFMQYGIVANYCWLLVEGLYLHNLLGLATLPERSFFSLYLGIGWGAPMLFVVPWAVVKCLFENVQCWTSNDNMGFWWILRFPVFLAILINFFIFVRIVQLLVAKLRARQMHHTDYKFRLAKSTLTLIPLLGVHEVVFAFVTDEHAQGTLRSAKLFFDLFLSSFQGLLVAVLYCFLNKEVQSELRRRWHRWRLGKVLWEERNTSNHRASSSPGHGPPSKELQFGRGGGSQDSSAETPLAGGLPRLAESPF

SEQ ID NO: 2 - protein, 128 aa, Homo sapiens
QVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSS

SEQ ID NO: 3 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIK

SEQ ID NO: 4 - protein, 128 aa, Homo sapiens
QVQLVESGGGVVQPGRSLRLSCAASGFTFSSYGMHWVRQAPGKGLEWVAVMWYDGSNKDYVDSVKGRFTISRDNSKNTLYLQMNSLRAEDTAVYYCAREKDHYDILTGYNYYYGLDVWGQGTTVTVSS

SEQ ID NO: 5 - protein, 107 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFVTYYCLQHNSNPLTFGGGTKVEIK

SEQ ID NO: 6 - protein, 128 aa, Homo sapiens; identical to SEQ ID NO: 2

SEQ ID NO: 7 - protein, 107 aa, Homo sapiens
DIQMTQSPSSLSASVGDRVTITCRASQGIRNDLGWYQQKPGKAPKRLIYAASSLESGVPSRFSGSGSGTEFTLTISSVQPEDFVTYYCLQHNSNPLTFGGGTKVEIK

SEQ ID NO: 8 - protein, 471 aa, Artificial Sequence
  heavy chain of a chimeric antibody that binds GCGR
MEFGLSWVFLVALLRGVQCQVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 9 - protein, 236 aa, Artificial Sequence
  light chain of a chimeric antibody that binds GCGR
MDMRVPAQLLGLLLLWFPGARCDIQMTQSPSSLSASVGDRVTITCRASQGIRNDLGWYQQKPGKAPKRLIYAASSLESGVPSRFSGSGSGTEFTLTISSVQPEDFVTYYCLQHNSNPLTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 10 - protein, 125 aa, Homo sapiens
QVQLVESGGGVVQPGRSLRLSCAASGFTFSNYGMHWVRQAPGKGLEWVAVILSDGRNKYYADSVKGRFTISRDNSKNTLYLQMNSLRAEDTAVYYCARDDYEILTGYGYYGMDVWGQGTTVTVSS

SEQ ID NO: 11 - protein, 125 aa, Homo sapiens
QVQLVESGGGVVQPGRSLRLSCAASGFTFSSYGMHWVRQAPGKGLEWVAVILNDGRNKYYADSVKGRFTISRDNSKNTLYLQMNSLRAEDTAVYYCARDDYEILTGYGYYGMDVWGQGTTVTVSS

SEQ ID NO: 12 - protein, 129 aa, Homo sapiens
QVQLQQSGPGLVKPSQTLSLTCAISGDSVSSNGAAWNWIRQSPSRGLEWLGRTYYRSKWYYDYAGSVKSRININPDTSKNQFSLQVNSVTPEDTAVYYCTRDRSSGWNEGYYYYGMDVWGQGTTVTVSS

SEQ ID NO: 13 - protein, 128 aa, Homo sapiens
QVQLVESGGGVVQPGRSLRLSCAASGFTFSSYDIHWVRQAPGKGLEWVAVLSSDGNNKYCADSVKGRFTISRDNSKNTLYLQMNSLRTEDTAVYYCAREEVYYDILTGYYDYYGMDVWGQGTTVTVSS

SEQ ID NO: 14 - protein, 126 aa, Homo sapiens
QVQLQESGPGLVKPSETLSLTCTVSGGSISTYFWTWIRQFPGKGLEWIGYIFYSGSTNYNPSLKSRVTISVDTSKNQFSLKLSSVTAADTAVYYCAREGYYDILTGEDYSYGMDVWGQGTTVTVSS

SEQ ID NO: 15 - protein, 129 aa, Homo sapiens
QVQLQQSGPGLVKPSQILSLTCAISGDRVSSNGAAWNWIRQSPSRGLEWLGRTYYRSKWYYDYAGSVKSRININPDTSKNQFSLQVNSVTPEDTAVYYCARDRSSGWNEGYYYYGMDVWGQGTTVTVSS

SEQ ID NO: 16 - protein, 126 aa, Homo sapiens
QVQLQESGPGLVKPSETLSLTCTVSGGSISTYFWTWIRQFPGEGLEWIGYIFYSGNTNYNPSLTSRVTISVDTSKNQFSLKLSSVTAADTAVYYCAREGYYDILTGEDYSYGIDVWGQGTTVTVSS

SEQ ID NO: 17 - protein, 125 aa, Homo sapiens
QVQLVESGGGVVQPGRSLRLSCAASGFIFSSYGMHWVRQAPGKGLEWVAVISNDGSNKYYADFVKGRFTISRDNSKNTLYLQMNSLRAEDTAVYYCAREDYDILTGNGVYGMDVWGQGTTVTVSS

SEQ ID NO: 18 - protein, 122 aa, Homo sapiens
EVQLVESGGGLVQPGGSLRLSCAASGFTFSSYTMNWVRQAPGKGLEWVSYISGSSSLIYYADSVKGRFTISRDNAKNSLYLHMNSLRDEDTAVYYCARARYNWNDYYGMDVWGQGTTVTVSS

SEQ ID NO: 19 - protein, 116 aa, Homo sapiens
QVQLVESGGGVVQPGRSLRLSCAASGFAFSSYGIHWVRQAPGKGLEWVAGIWYDGSNKYYADSVKGRFTVSRDNSKNTLYLQMNSLRAEDTAVYYCARLFDAFDIWGQGTMVTVSS

SEQ ID NO: 20 - protein, 128 aa, Homo sapiens
EVQLVESGGGLVQPGGSLRLSCAASGFIFSSYTMNWVRQAPGKGLEWVSYISSSSSLIYYADSVKGRFTISRDNAKNSLYLQMNSLRDEDTAVYYCARSDYYGSGSYYKGNYYGMDVWGQGTTVTVSS

SEQ ID NO: 21 - protein, 128 aa, Homo sapiens
QVQLVESGGGVVQPGRSLRLSCAASGFTFSSYGMHWVRQAPGKGLEWVTIIWSDGINKYYADSVKGRFTISRDNSKNTLNLQMNSLRAEDTAVYYCARERGLYDILTGYYDYYGIDVWGQGTTVTVSS

SEQ ID NO: 22 - protein, 128 aa, Homo sapiens; identical to SEQ ID NO: 21

SEQ ID NO: 23 - protein, 116 aa, Homo sapiens
EVQLVESGGGLVKPGGSLRLSCAASGITFRSYSMNWVRQAPGKGLEWVSAISSSSSYIYYADSVKGRFTISRDNAKNSVYLQMNSLRAEDTAVYYCARGRYGMDVWGQGTTVTVSS

SEQ ID NO: 24 - protein, 125 aa, Homo sapiens
QVQLVESGGGVVQPGRSLRLSCAASGSTFRSYDMHWVRQAPGKGLEWVAVISYDGSNKYYGDSVKGRLTISRDNSKNTLYLQMNSLRAEDTAVYYCARDQYDILTGYSSDAFDIWGQGTMVTVSS

SEQ ID NO: 25 - protein, 127 aa, Homo sapiens
QVQLVESGGGVVQPGRSLRLSCAASGFTFSRYGMHWVRQAPGKGLEWVAVIWYDGSHKYYEDSVKGRFTISRDNSKNTLYLQMNSLRADDTGVYYCARVGYGSGWYEYYYHYGMDVWGQGTTVTVSS

SEQ ID NO: 26 - protein, 128 aa, Homo sapiens; identical to SEQ ID NO: 2

SEQ ID NO: 27 - protein, 128 aa, Homo sapiens; identical to SEQ ID NO: 2

SEQ ID NO: 28 - protein, 116 aa, Homo sapiens
QVQLVESGGGVVQPGRSLRLSCAASGITFSSYGMHWVRQAPGKGLEWVASIWYDGSNKYYVDSVKGRFTIFRDNSKKTLYLQMNRLRAEDTAVYYCARLGGGFDYWGQGTLVTVSS

SEQ ID NO: 29 - protein, 108 aa, Homo sapiens
DIQMTQSPSSLSASVGDRVTITCRASQDISNYLAWFQKKPGKAPKSLIYVVSSLQSGVPSRFSGSGSGTDFTLTINNLQPEDFATYYCQQYNHYPLTFGGGTRVEIKR

SEQ ID NO: 30 - protein, 108 aa, Homo sapiens
DIQMTQSPSSLSASVGDRVTITCRASQDISNYLAWFQQRPGKAPKSLIYVVSSLQSGVPSRFSGSGSGTDFTLTISNLQPEDFATYFCQQYNHYPLTFGGGTKVEIKR

SEQ ID NO: 31 - protein, 108 aa, Homo sapiens
DIQMTQFPSSLSASIGDRVTITCQASQDISNFLNWFQQKPGKAPKLLIYDASDLETGVPSRFSGSGAGTDFTFTISSLQPEDIATYFCQQYDDLPLTFGGGTRVDIKR

SEQ ID NO: 32 - protein, 108 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSNPLTFGGGTKVEIKR

SEQ ID NO: 33 - protein, 109 aa, Homo sapiens
QNVLTQSPGTLSLSPGERVTLSCRASQSVSSSYLAWYQQKPGQAPRLLIFGVSSRATGIPDRFSGSGSGTDFSLTISRLEPEDFAVYYCQQYGNSPFTFGPGTKVDIKR

SEQ ID NO: 34 - protein, 108 aa, Homo sapiens
DIQMTQFPSSLSASIGDRVTITCQASQDISNFLNWFQQKPGKAPKLLIYDASDLETGVPSRFSGSGAGTDFTFTISSLQPEDVATYFCQQYDNLPLTFGGGTKVDIKR

SEQ ID NO: 35 - protein, 109 aa, Homo sapiens
ENVLTQSPGTLSLSPGERATLSCRASQSVTSSYLAWYQQKPGQAPRLLIFGVSSRATGIPDRFSGSGSGTDFSLTISRLEPEDFAVYYCQQYGNSPFTFGPGTKVDIKR

SEQ ID NO: 36 - protein, 108 aa, Homo sapiens
DIQMTQSPSSLSASVGDRVTITCRASQGIDMYLAWFQQKPGKAPKSLIYAASSLQSGVPSKFSGSGFGTDFTLTISSLQPEDFATYYCQQYNIFPFTFGPGTKVDVKR

SEQ ID NO: 37 - protein, 108 aa, Homo sapiens
DIQMTQSPSSLSASVGDRVTITCRASQGIRNDLGWYQQKPGKAPKRLIYAASSLESGVPSRFSGSGSGTEFTLTISSLQPEDFATYYCLQHNSYPWTFGQGTKVEIKR

SEQ ID NO: 38 - protein, 114 aa, Homo sapiens
KIVMTQTPLALPVIPGEPASISCRSSQSLVDSDDGDTYLDWYLQKPGQSPQVLIHRLSYRASGVPDRFSGSGSGTDFTLKISRVEAEDVGIYYCMHRIEFPFTFGGGTKVEIKR

SEQ ID NO: 39 - protein, 108 aa, Homo sapiens
DIQMTQSPSSLSASVGDRVTITCRASQGIRNDLGWYQQRPGKAPKRLIYAASSLQTGVPSRFSGSGSGTEFTLTISSLQPEDFATYYCLQHNSYPWTFGQGTKVEIKR

SEQ ID NO: 40 - protein, 113 aa, Homo sapiens
GIVLTQSPLSLPVTPGEPASISCRSSQSLLHSNGYNYLDWYLQKPGQSPQLLIYLGSNRASGVPDRFSGSGSGTDFTLKISRVEAEDVGVYYCMEALQTMCSFGQGTKLEIKR

SEQ ID NO: 41 - protein, 113 aa, Homo sapiens
GIVLTQSPLSLPVTPGEPASISCRSSQSLLHSNGYNYLDWYLQKPGQSPQLLIYLGSNRASGVPDRFSGSGSGTDFTLKISRVEAEDVGVYYCMEALQTMSSFGQGTKLEIKR

SEQ ID NO: 42 - protein, 114 aa, Homo sapiens
DIVMTQTPLFLPVTPGEPASISCRSSQTLLDSDDGNTYLDWYLQKPGQSPQRLIYTLSYRASGVPDRFSGSGSGTDFTLKISRVEAEDVGIYYCMQHIEFPSTFGQGTRLEIKR

SEQ ID NO: 43 - protein, 107 aa, Homo sapiens
SYELTQPPSVSVSPGQTASITCSGDKLGDKYASWYQQKPGQSPVLVIYQSTKRPSGIPERFSGSNSGNTATLTISGTQAMDEADYYCQAWDSSTVVFGGGTKLTVLG

SEQ ID NO: 44 - protein, 113 aa, Homo sapiens
NIVMTQTPLSLSVTPGQPASISCKSSQSLLHSDGKNYLFWYLQKPGQSPQLLIYEVSYRFSGVPDRFSGSGSGTDFSLKISRVEAEDVGVYYCMQNIQPPLTFGQGTRLEIKR

SEQ ID NO: 45 - protein, 108 aa, Homo sapiens
DIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKR

SEQ ID NO: 46 - protein, 108 aa, Homo sapiens
DIQMTQSPSSLSASVGDRVTITCRASQGIRNDLGWYQQKPGKAPKRLIYAASSLESGVPSRFSGSGSGTEFTLTISSVQPEDFVTYYCLQHNSNPLTFGGGTKVEIKR

SEQ ID NO: 47 - protein, 114 aa, Homo sapiens
DIVLTQTPLSLPVTPGEPASISCRSSQSLLDRDDGDTYLDWYLQKPGQSPQLLIYTLSYRASGVPDRFSGSGSGTDFSLKISRVEAEDVGVYYCMQRIEFPFTFGPGTKVDIKR

SEQ ID NO: 48 - protein, 107 aa, Homo sapiens [sequence omitted]

SEQ ID NO: 49 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 50 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 51 - protein, 454 aa, Homo sapiens
QVQLVESGGGVVQPGRSLRLSCAASGFTFSSYGMHWVRQAPGKGLEWVAVMWYDGSNKDYVDSVKGRFTISRDNSKNTLYLQMNRLRAEDTAVYYCAREKDHYDILTGYNYYYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 52 - protein, 214 aa, Homo sapiens
DIQMTQSPSSLSASVGDRVTITCRASQGIRNDLGWYQQKPGKAPKRLIYAASSLQSGVPSRFSGSGSGTEFTLTISSVQPEDFVTYYCLQHNSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC